SEQUENCE LISTING (22 sequences)

SEQ ID NO: 1 - protein, 281 aa, Streptococcus pyogenes
MSFKKWIGIVGIALASTVLVACSEKQDDKNTLTIGVMTKTESDQARWDKVEELLKKDNITLKYKEFTDYSQPNKAVANGEVDINAFQHYNFLNNWNKENKEHLVAIADTYISPINLFSGTSQDGKAKYKSVADLPNGAQIAVPNDATNESRALYVLQSAGLIKLDVSGDQLATIANISENKKKLDIKELDASQTARALVSADAAVVNNSYAVPAKIDYKTSLFKEKADDNSKQWINIIAGQKDWEKSEKADAIKKLIKAYQTDEVKKVVEKTSNGIDVSVW

SEQ ID NO: 2 - protein, 281 aa, Streptococcus pyogenes; identical to SEQ ID NO: 1

SEQ ID NO: 3 - protein, 280 aa, Streptococcus pyogenes
MKLKKILGLLGLALAAAFVLTACGQKNDKNTLTVGVMTMTDSDKERWDKIEELLKKENIKLKFKEFTDYSQPNKALKNGEIDINSFQHYNFLNNWNKENKGDLVTVAETYISPINLFSGTENGKAKYSSAKEIPNGGQIAIPNDATNESRALYVLQDAGLIKLNVSGDELATVKNIKSNPKNLDIKEVDASQTARNLASVDAAVVNNSYAVPAKIDFKTSLYKEKVNEGSKQWINIIAAQKNWKKSKKAAAIKKLIKAYHTDAVKKVIKKTAKGVDEPVW

SEQ ID NO: 4 - protein, 280 aa, Streptococcus pyogenes; identical to SEQ ID NO: 3

SEQ ID NO: 5 - protein, 721 aa, Streptococcus pyogenes
MITIKNPKILKWLKYVLSAILSLIILVIIIGGLLFTFYISSAPKLSEAQLKSTNSSLVYDGNNNLIADLGSEKRENVTADSIPINLVNAITSIEDKRFFNHRGVDLYRIFGAAFHNLTSQTTQGGSTLDQQLIKLAYFSTNESDQTLKRKAQEVWLALQMERKYTKQEILTFYINKVYMGNGNYGMLTAAKSYYGKDLKDLSYAQLALLAGIPQAPSQYDPYLHPEAAQNRRNVVLQQMYMEKHLTKAEYETAIATPVAEGLQSLQQRSTYPKYMDNYLKQVIEEVKKETNKDIFTAGLKVYTNIIPDAQQTLYNIYHSGDYVYYPDQDFQVASTIVDVTNGHVIAQLGGRNQDENVSFGTNQAVLTDRDWGSTMKPITAYAPAIESGVYTSTAQSTNDSVYYWPGTTTQLFNWDLRYNGWMTIQAAIMLSRNVPAVRALEAAGLDYARSFLSSLGINYPEMHYSNAISSNNSSSDKKYGASSEKMAAAYAAFANGGIYHKPRYVNKVEFSDGTSKTFDEKGKRAMKETTAYMMTDMLKTVLTYGTGTAAAIPGVAQAGKTGTSNYTDEELAKIGEKYGLYPDYVGTLAPDENFVGFTKRYAMAVWTGYKNRLTPVYGSSLEIASDVYRSMMTYLTNGYSEDWTMPNGLYRSGGFLYLSGTYASNTDYTNSVYNNLYSNNTTTASSQTTSDDTSSSNDTSNSTNTDNNGSHPSTDDKKTTH

SEQ ID NO: 6 - protein, 721 aa, Streptococcus pyogenes; identical to SEQ ID NO: 5

SEQ ID NO: 7 - protein, 126 aa, Streptococcus pyogenes
MKTIRRYDVNEDRGHTGLVEAGDFYYLNYCVGNVGQDIESQINGAFDEMERRLALVGLTLDAVVQMDCLFRDVWNIPVMEKMIKERFNGRYPARKSIQTEFAHHGGPQGLLFQVDGVAYSKHISMT

SEQ ID NO: 8 - protein, 126 aa, Streptococcus pyogenes; identical to SEQ ID NO: 7

SEQ ID NO: 9 - protein, 306 aa, Streptococcus pyogenes
MSNEFINFEKISRESWKTLHQKAKALLTQEELKSITSLNDNISINDVIDIYLPLINLIQVYKIAQENLSFSKSLFLKKDIQLRPFIIGISGSVAVGKSTTSRLLQLLLSRTHPNSQVELVTTDGFLYPNQFLIEQGLLNRKGFPESYNMELLLDFLDSIKNGQTAFAPVYSHDIYDIIPNQKQSFNNPDFLIVEGINVFQNQQNNRLYMSDYFDFSIYIDADSSHIETWYIERFLSILKLAKRDPHNYYAQYAQLPRSEAIAFARNVWKTVNLENLEKFIEPTRNRAELILHKSADHKIDEIYLKK

SEQ ID NO: 10 - protein, 306 aa, Streptococcus pyogenes
MANEFINFEKISRKTWQHLHQESQPPLNENELNSIKSLNDRISIKDVTDIYLPLISLIQIYKKSQENLSFSKSIFLQKNISNRPFIIGVSGSVAVGKSTTSRLLQLLLARTFKDSSVELMTTDGFLYPNAVLSSRHMLNKKGFPESYDMERLLDFLDTIKNGQSAEIPVYSHEIYDIVPNKSQIIEVPDFLIIEGINVFQNPQNNRLYMSDFFDFSIYIDADSDYIENWYLERFATLLDLAKNDKQNYYNRFLKLGEKGALDFARDIWKDINLVNLEKYIEPTRSRAELILHKTKNHKIDEIYLKK

SEQ ID NO: 11 - protein, 306 aa, Streptococcus pyogenes
MNNEFINFDRISRENWKDLHQQSQALLTEKELESIKSLNDNINIQDVIDIYLPLINLIQIYKRSQENLSFSKAIFLKKENYQRPFIIGISGSVAVGKSTTSRLLQLLISRTFKDSHVELVTTDGFLYPNEKLIQNGILNRKGFPESYDMESLLNFLDTIKNGITAKIPIYSHEIYDIVPNQLQTIETPDFLILEGINVFQNQQNHRLYMNDYFDFSIYIDAENKQIEEWYLQRFNSLLQLAEADPSNYYHKFTQIPPHKAMELAKDIWKTINLVNLEKYIEPTRNRADFIIHKGKHHKIDEIYLKK

SEQ ID NO: 12 - protein, 306 aa, Streptococcus pyogenes; identical to SEQ ID NO: 10

SEQ ID NO: 13 - protein, 306 aa, Streptococcus pyogenes; identical to SEQ ID NO: 9

SEQ ID NO: 14 - protein, 244 aa, Streptococcus pyogenes
MIEFKHVSKLYGDKEALSDLNVTINDGEIFGLIGHNGAGKTTTISILTSIIEASYGEVFVDGQLLTENREAIKKQIAYVPDSPDIFLNLTPNEYWQFLAKIYGVSDEDREERLAQLTTLFELKEEVNQTIDSFSHGMRQKVIVIGALVSNPNIWILDEPLTGLDPQASFDLKEMMKAHAASGHTVLFSTHVLSVAEQLCDRIGILKKGKLIFVGTIDELKEHHPDKDLESIYLELAGRKAQEEG

SEQ ID NO: 15 - protein, 300 aa, Streptococcus pyogenes
MTQQSMLLSIEQVNKSYGKNQVLSDISFDIYKGEICGLVGQNGAGKTTLMRILSGLIGKDSGQIKQLQPYRMGSIIESPTLYPNMTAHDNLYYAALQLRLADAKERIHEVLELIGLEKVSKKKKVKDYSLGMRQRLAIGLSILDFPEFLILDEPINGLDPAGIKEMRQIILNLRDCYGITILISSHILSELDLVVDRYVIMHKGKVIKSMDKAELKAQVKVQIALHTSDDQLVKNRLLELGLRVETDGQMLLIKPTLSVMELIKIVLDLPVEIFDIYHHQVSFEHYYLDLLGKDAASPLI

SEQ ID NO: 16 - protein, 300 aa, Streptococcus pyogenes; identical to SEQ ID NO: 15

SEQ ID NO: 17 - protein, 42 aa, Streptococcus pyogenes
MKIKGDSKEKKEGVVFLFCHTNIIFEIGAVCHLGVVIVFRPR

SEQ ID NO: 18 - protein, 76 aa, Streptococcus pyogenes
MTFFFIRPPFSITFKQDYLITLRKRLQDMPHFNLEHSSNNDYHPLIGSQKLNSQNEFPLAWLLGKLKSHTTARPSV

SEQ ID NO: 19 - protein, 447 aa, Streptococcus pyogenes
MASNHMNEQEREQAKFSLSGATLYGINAVIGSGIFLLPRAIYKGLGPASIAVMFGTAILTIMLAVCFAEVSGYFGKNGGAFQYSKRAFGDFIGFNVGFLGWTVTIFAWAAMAAGFARMFIITFPAFEGWHIPLSIGLIILLSLMNIAGLKTSKIVTITATIAKLIPIVAFCACTLFFIKNGLPNFTPFVQLEPGTNLLGAISNTAVYIFYGFIGFETLSIVAGEMRDPEKNVPRALLGSISIVSVLYMLIIGGTIAMLGSQIMMTNAPVQDAFVKMIGPAGAWMVSIGALISITGLNMGESIMVPRYGAAIADEGLLPAAIAKQNQNGAPLVAILVSGAIAIVLLLTGSFENLAKLSVVFRFFQYIPTALAVMKLRKDAPDANVIFRVPFGPIIPILAVIVSLVMIWGDNPMNFVYGAVGVIIASSVYYLMHGRSHQMDQMPPKEQI

SEQ ID NO: 20 - protein, 42 aa, Streptococcus pyogenes; identical to SEQ ID NO: 17

SEQ ID NO: 21 - protein, 76 aa, Streptococcus pyogenes; identical to SEQ ID NO: 18

SEQ ID NO: 22 - protein, 447 aa, Streptococcus pyogenes; identical to SEQ ID NO: 19

(19) United States
(12) Patent Application Publication
(10) Pub. No.: US 2011/0150916 A1
(43) Pub. Date: Jun. 23, 2011

(54) COMPOSITIONS AND METHODS FOR TREATMENT OF GROUP A STREPTOCOCCI

(75) Inventors: Kowthar Salim, Etobicoke (CA); Joyce De Azavedo, Toronto (CA); Dennis Cvitkovitch, Oakville (CA)

(73) Assignee: MOUNT SINAI HOSPITAL, Toronto (CA)

(21) Appl. No.: 11/940,602

(22) Filed: Nov. 15, 2007

Related U.S. Application Data
(60) Provisional application No. 60/859,320, filed on Nov. 16, 2006.

(51) Int. Cl. A61K 39/09; C07K 14/315; C07K 7/06; C07K 7/08; G01N 33/68; G01N 33/00; G01N 33/53; A61P 37/04; A61P 31/04

(52) U.S. Cl. 424/190.1; 530/359; 530/330; 530/329; 530/328; 530/327; 530/326; 530/325; 530/324; 436/86; 436/94; 436/501

(57) ABSTRACT

Immunogenic compositions and vaccines are described comprising GAS Markers including AtmB Proteins. Methods for detecting GAS diseases in a subject are also described comprising measuring GAS markers or antibodies against GAS markers in a sample from the subject. The invention further provides kits for carrying out the methods of the invention and therapeutic applications for GAS diseases employing GAS markers, polynucleotides encoding the markers, and/or binding agents for the markers.

### CROSS-REFERENCE TO RELATED APPLICATIONS

This application claims the benefit of the priority of U.S. Provisional Patent Application No. 60/859,320, filed Nov. 16, 2006.

### BACKGROUND OF THE INVENTION

The invention relates to compositions and methods for the diagnosis, treatment, prevention and amelioration of diseases caused by group A Streptococcus.

Group A Streptococcus (GAS), also known as Streptococcus pyogenes, can cause several types of diseases in humans, including strep throat, scarlet fever, impetigo, cellulitis-erysipelas, rheumatic fever, acute glomerular nephritis, endocarditis, and necrotizing fasciitis and it is associated with significant morbidity and mortality worldwide (Carapetis, J. R., et al., 2005 Lancet Infect. Dis. 5:685-694). The development of effective and safe vaccines against streptococcal infections has been ongoing (Bisno, A. L., et al., 2005 Clin. Infect. Dis. 41:1150-1156). A useful vaccine against GAS would reduce health care costs and numerous physician visits.

A number of group A Streptococcus vaccine candidates have been identified, such as M proteins (Bessen, D. et al; Fischetti, V. A. 1989 Infect. Immun. 64:1495-1501; Lancefield, R. C. 1962, J. Immun. 89:307-313), C5a peptidase (Cleary, P. P., Matsuka, Y. V. et al; Kapur, V. et al. 1994 Infect Immun. 65:2080-2087), cysteine protease (Dale, J. B., et al, Microb. Pathogenesis. 16:443450) and lipoteichoic acid (Dale, J. B., et al., 1996 J. Infect. Dis. 169:319-323; Lancefield, R. C. 1962; Clin. Microbiol. 2:285-314). None of the potential vaccine candidates has shown a clear-cut advantage in conferring immunity to streptococcal disease prevention. For example, there are difficulties associated with a vaccine strategy involving the M protein, such as the large number of serologic M types, and the observation that some M proteins contain epitopes that cross-react with human tissues. Thus, a need still exists for a flexible, effective, multivalent vaccine against GAS that includes several determinants which would provide protection against disease. Furthermore, given the speed of development of severe invasive disease, there is a need for rapid diagnosis of GAS infection which does not depend on conventional culture methods.

### SUMMARY OF THE INVENTION

Applicants constructed in-frame allelic replacement atmB mutants in two M serotype strains (M1 and M49) of S. pyogenes. These mutants were assessed in vitro in a whole blood killing assay and were found to be significantly attenuated in their capacity to survive in blood relative to the wild-type parent strains. A quantitative analysis of the total cell-associated and released hyaluronic acid capsule indicated that the mutants produced significantly more capsule than their respective parent strains. The mutants were also severely attenuated in virulence in vivo utilizing two murine models of invasive S. pyogenes infections. In the soft-tissue infection model the M49 serotype strain mutant did not form lesions in mice and only one mouse infected with the M1 serotype atmB mutant formed a lesion. Furthermore, in the septicemic model the mutants were not only cleared from infected tissues but the mice survived unlike those infected with the wild-type parent strains that died within 24 h. These results demonstrate that atmB is an important virulence factor of S. pyogenes that contributes significantly to the ability of this organism to cause invasive disease.

An immunogenic composition for protecting subjects against infection by group A Streptococcus is provided. The immunogenic composition comprises an immunogenic amount of a region of a group A Streptococcus atmB putative lipoprotein. In one composition, the region of a group A Streptococcus atmB putative lipoprotein defines an epitope which induces the formation of bactericidal antibodies against GAS. In one aspect, the region of a group A Streptococcal atmB putative lipoprotein is immunoreactive and found in the most prevalent GAS serotypes associated with a selected disease.

The region of a group A Streptococcus atmB putative lipoprotein present in the immunogenic compositions may be in the form of a polypeptide or part of a lipoprotein. Thus, in one aspect, the immunogenic composition comprises a polypeptide encoded by atmB (e.g. GeneID.:1008547 and Accession Nos. AAM78840 or NP_{—}664037), or a portion, isoform, homolog, variant, or precursor of the polypeptide, including modified forms of the polypeptide and derivative (hereinafter defined as “AtmB Protein”). An immunogenic AtmB Protein may also be a chimeric or fusion polypeptide or conjugate. In another aspect, the immunogenic composition comprises a lipoprotein comprising a polypeptide encoded by atmB.

In one embodiment, an immunogenic composition comprises synthetic peptides about to 200, 10 to 150, 10 to 100, 20 to 100, 10 to 50 or 20 to 25 amino acids in length which are portions of an AtmB Protein. In embodiments, the synthetic peptides are serotype specific peptides. Synthetic peptides may be used, for example, individually, in a mixture, or in a polypeptide or protein. For example, a polypeptide or protein can be created by fusing or linking the peptides to each other, synthesizing the polypeptide or protein based on the peptide sequences, and linking or fusing the peptides to a backbone. In addition, a liposome may be prepared with the peptides conjugated to it or integrated within it.

An immunogenic composition may also comprise one or more regions of other group A Streptococcus antigens including without limitation regions of polypeptides such as penicillin-binding proteins (PBPs) (e.g., pbp1A), TdcF (e.g. S. pyogenes hypothetical protein spyM18_{—}2121), CoaA Proteins, Spy1674 Proteins, Spy1784 Proteins and/or Spy1733 Proteins, especially Pbp1A Proteins and/or TdcF Proteins.

The immunogenic compositions are preferably recognized by AtmB Protein specific antibodies and are capable of eliciting functional opsonic antibodies and/or anti-attachment antibodies without eliciting tissue cross-reactive antibodies.

The immunogenic compositions may be useful for raising antibodies which have application for prophylactic and diagnostic purposes. Therefore, also provided are isolated antibodies that specifically bind to an AtmB Protein, and in particular antibodies elicited in response to an immunogenic composition or vaccine described herein. An antibody may be a monoclonal or polyclonal antibody or an antibody fragment (e.g., Fab or F(ab′)₂ fragment). In one aspect, antibodies specific for an AtmB Protein that can be used therapeutically to destroy or inhibit a GAS disease or to block AtmB associated with a GAS disease are provided. In another aspect, AtmB Proteins may be used in various immunotherapeutic methods to promote immune-mediated destruction or inhibition of GAS expressing AtmB Proteins. In a further aspect, compositions are provided comprising antibodies specific for one or more AtmB Protein, peptides derived therefrom, or chemically produced (synthetic) peptides, and optionally one or more penicillin-binding proteins (PBPs) (e.g., pbp1A), TdcF Proteins, CoaA Proteins, Spy1674 Proteins, Spy1784 Proteins and Spy1733 Proteins, especially Pbp1A Proteins and/or TdcF Proteins, peptides derived therefrom, or chemically produced (synthetic) peptides, and a pharmaceutically acceptable carrier, excipient, or diluent.

An immunogenic composition described herein may be useful as a vaccine and the invention contemplates a vaccine comprising the immunogenic composition.

In an aspect, vaccines are contemplated for stimulating or enhancing in a subject to whom the vaccine is administered production of antibodies directed against an. AtmB lipoprotein, AtmB Protein, peptides derived therefrom, or chemically produced (synthetic) peptides, or any combination of these molecules and optionally penicillin-binding proteins (PBPs) (e.g., pbp1A), TdcF Proteins, CoaA Proteins, Spy1674 Proteins, Spy1784 Proteins and/or Spy1733 Proteins, especially Pbp1A Proteins and/or TdcF Proteins, peptides derived therefrom, or chemically produced (synthetic) peptides, or any combination of these molecules.

The immunogenic composition may be capable of eliciting active and passive protection against infection by group A Streptococcus. For passive protection, immunogenic antibodies can be produced by immunizing a subject with a vaccine comprising an immunogenic composition described herein and then recovering the immunogenic antibodies from the subject. Thus, a composition is contemplated for passive immunization comprising antibodies specific for AtmB Proteins.

In one aspect, the immunogenic composition or vaccine may be used to inhibit or reduce the growth of group A Streptococcal bacteria, in particular, S. pyogenes, in blood and/or reduce phagocytic resistance. Accordingly, the use of an atmB lipoprotein, AtmB Proteins, peptides derived therefrom, or chemically produced (synthetic) peptides, or any combination of these molecules, for use in the preparation of vaccines to prevent a GAS disease and/or to treat a GAS disease is contemplated herein.

An immunogenic composition or vaccine may further comprise additional components, including but not limited to, carriers, diluents, excipients, vehicles (e.g., encapsulated, liposomes), and other immune-stimulatory molecules (e.g., adjuvants, other vaccines). In an aspect, a vaccine further comprises an adjuvant such as aluminum hydroxide, aluminum phosphate, monophosphoryl lipid A, QS21 or stearyl tyrosine.

A polypeptide or lipoprotein in an immunogenic composition or vaccine may be conjugated to a native or recombinant bacterial protein such as tetanus toxoid, cholera toxin, diphtheria toxoid, or CRM₁₉₇.

Additionally contemplated is a DNA vaccine comprising DNA encoding an AtmB Protein or synthetic peptides thereof, and optionally a penicillin-binding protein (PBPs) (e.g., pbp1A), TdcF Protein, CoaA Protein, Spy1674 Protein, Spy1784 Protein and/or Spy1733 Protein, or synthetic peptides thereof.

In an aspect, methods of immunizing a mammal against infection by group A Streptococcus is provided and includes administering an immunogenic amount of a composition of the invention. In another aspect, the immunogenic composition is used to provide protection against infection by group A Streptococcus in those populations most at risk of contracting GAS infections and disease namely adults, pregnant women and, in particular, infants and children.

A method for treating or preventing a GAS disease in a patient is also provided comprising administering to a patient in need thereof antibodies specific for one or more AtmB Proteins associated with a GAS disease, and optionally penicillin-binding proteins (PBPs) (e.g., pbp1A), TdcF Proteins, CoaA Proteins, Spy1674 Proteins, Spy1784 Proteins and/or Spy1733 Proteins, especially Pbp1A Proteins and/or TdcF Proteins, or a composition described herein. Also provided is a method for stimulating or enhancing in a subject production of antibodies directed against one or more AtmB Protein, and optionally penicillin-binding proteins (PBPs) (e.g., pbp1A), TdcF Proteins, CoaA Proteins, Spy1674 Proteins, Spy1784 Proteins and/or Spy1733 Proteins, especially Pbp1A Proteins and/or TdcF Proteins. The method comprises administering to the subject a vaccine described herein in a dose effective for stimulating or enhancing production of the antibodies.

Further provided is a method for treating, preventing, or delaying recurrence of a GAS disease. The method comprises administering to the subject a composition or vaccine described herein in a dose effective for treating, preventing, or delaying recurrence of a GAS disease.

In further aspects, methods for using the immunogenic compositions, vaccines, or antibodies are provided, as are methods for tailoring vaccines. In another aspect, methods for using the immunogenic compositions, vaccines, or antibodies in the preparation of a medicament for treating a GAS disease are provided.

Also provided are markers and marker sets that distinguish group A Streptococcus diseases (GAS diseases). A marker set may comprise or consist of a plurality of polypeptides and/or polynucleotides encoding one or more of an AtmB Protein, penicillin-binding proteins (PBPs) (e.g., pbp1A), TdcF Proteins, CoaA Proteins, Spy1674 Proteins, Spy1784 Proteins and Spy1733 Proteins, especially Pbp1A Proteins and/or TdcF Proteins. GAS markers and marker sets can be used for diagnosis, monitoring (i.e. monitoring progression or therapeutic treatment), prognosis, treatment, or classification of a GAS disease.

The levels of markers or marker sets in a sample may be determined by methods as described herein and generally known in the art. A marker set may be used to detect antibodies in patient samples in order to diagnose a GAS disease.

In one aspect, a method is provided for characterizing or classifying a patient sample comprising detecting a difference in the expression of a first plurality of GAS markers relative to a control, the first plurality of GAS markers consisting of one or more AtmB Proteins and polynucleotides encoding AtmB Proteins, and optionally penicillin-binding proteins (PBPs) (e.g., pbp1A) and polynucleotides encoding PBPs, TdcF Proteins and polynucleotides encoding TdcF Proteins, CoaA Proteins and polynucleotides encoding CoaA Proteins, Spy1674 Proteins and polynucleotides encoding Spy1674 Proteins, Spy1784 Proteins and polynucleotides encoding Spy1784 Proteins, and/or Spy1733 Proteins and polynucleotides encoding Spy1733 Proteins, especially Pbp1A Proteins and/or TdcF Proteins.

In another aspect, a method is provided for diagnosing a GAS disease in a subject and comprises:
(a) obtaining a sample from a subject;
(b) detecting in the sample one or more GAS markers wherein the GAS markers comprise group A Streptococcus atmB putative lipoprotein or polynucleotides encoding a group A Streptococcus atmB putative lipoprotein, and optionally one or more of penicillin-binding proteins (PBPs), polynucleotides encoding PBPs, TdcF, polynucleotides encoding TdcF, CoaA, polynucleotides encoding CoaA, Spy1674, polynucleotides encoding Spy1674, Spy1784, polynucleotides encoding Spy1784, Spy1733, and polynucleotides encoding Spy1733; and
(c) comparing the detected amounts with amounts detected for a standard.

In a further aspect, a method is provided for detecting, in particular diagnosing, a GAS disease in a patient and comprises:
(a) obtaining a sample from a patient;
(b) detecting in the sample at least one GAS marker comprising or consisting of an AtmB Protein or polynucleotide encoding AtmB Protein and optionally a penicillin-binding protein (PBPs) (e.g., pbp1A) or polynucleotide encoding PBP, and a TdcF Protein or polynucleotide encoding a TdcF Protein; and
(c) comparing the detected amounts with amounts detected for a standard.

The term “detect” or “detecting” includes assaying or otherwise establishing the presence or absence of the target markers, subunits thereof, or combinations of reagent bound targets, antibodies and the like, or assaying for, ascertaining, establishing, or otherwise determining one or more factual characteristics of a GAS disease. The term encompasses diagnostic, prognostic, and monitoring applications for the markers.

In still a further aspect, a method of assessing whether a patient is afflicted with or has a pre-disposition for a GAS disease is provided and comprises comparing:
(a) levels of GAS protein markers or GAS polynucleotide markers associated with a GAS disease in a sample from the patient; and
(b) normal levels of GAS disease markers in samples of the same type obtained from control patients not afflicted with the disease, wherein altered levels of the markers relative to the corresponding normal levels of markers is an indication that the patient is afflicted with a GAS disease.

In an aspect of a method described herein for assessing whether a patient is afflicted with or has a pre-disposition for a GAS disease, higher levels of the markers in a sample relative to the corresponding normal levels is an indication that the patient is afflicted with or has a pre-disposition for a GAS disease.

In another aspect of a method described herein for assessing whether a patient is afflicted with or has a pre-disposition for a GAS disease, lower levels of GAS disease markers in a sample relative to the corresponding normal levels is an indication that the patient is afflicted with a GAS disease.

In a further aspect, a method for screening a subject for a GAS disease is provided comprising (a) obtaining a biological sample from a subject; (b) detecting the amount of GAS markers in the sample; and (c) comparing the amount of markers detected to a predetermined standard, where detection of a level of markers that differs significantly from the standard indicates a GAS disease.

In an embodiment, a significant difference between the levels of GAS marker levels in a patient and normal levels is an indication that the patient is afflicted with or has a predisposition to a GAS disease.

In a further embodiment, the amount of GAS marker(s) detected is greater than that of a standard and is indicative of a GAS disease. In another embodiment, the amount of GAS marker(s) detected is lower than that of a standard and is indicative of a GAS disease.

In an embodiment the GAS marker is an AtmB Protein and the amount of AtmB Protein detected is greater than a standard determined from a sample from an uninfected individual.

A method is therefore provided for detecting, in particular diagnosing, a GAS disease in a patient, the method comprising:
(a) obtaining a sample from a patient;
(b) detecting in the sample antibodies against GAS protein markers, in particular an AtmB Protein, and optionally antibodies against one or more of penicillin-binding proteins (PBPs) (e.g., pbp1A), TdcF Proteins, CoaA Proteins, Spy1674 Proteins, Spy1784 Proteins and Spy1733 Proteins, especially Pbp1A Proteins and/or TdcF Proteins; and
(c) comparing the detected amount with an amount detected for a standard.

Also provided is a method of assessing whether a patient is afflicted with or has a pre-disposition for a GAS disease, the method comprising comparing:
(c) levels of antibodies against GAS protein markers in a sample from the patient; and
(d) normal levels of antibodies against GAS protein markers in samples of the same type obtained from control patients not afflicted with the disease, wherein altered levels of the antibodies relative to the corresponding normal levels of antibodies is an indication that the patient is afflicted with a GAS disease.

In an aspect of a method described herein for assessing whether a patient is afflicted with or has a pre-disposition for a GAS disease, higher levels of the antibodies in a sample relative to the corresponding normal levels is an indication that the patient is afflicted with or has a pre-disposition for a GAS disease.

In another aspect of a method described herein for assessing whether a patient is afflicted with or has a pre-disposition for a GAS disease, lower levels of antibodies in a sample relative to the corresponding normal levels is an indication that the patient is afflicted with a GAS disease.

In a further aspect, a method for screening a subject for a GAS disease is provided comprising (a) obtaining a biological sample from a subject; (b) detecting the amount of antibodies against GAS markers in the sample; and (c) comparing the amount of antibodies detected to a predetermined standard, where detection of a level of antibodies that differs significantly from the standard indicates a GAS disease.

In an embodiment, a significant difference between the levels of antibodies against GAS markers in a patient and normal levels is an indication that the patient is afflicted with or has a predisposition to a GAS disease.

In a particular embodiment the amount of antibodies detected is greater than that of a standard and is indicative of a GAS disease. In another particular embodiment, the amount of antibodies detected is lower than that of a standard and is indicative of a GAS disease.

In an embodiment the antibodies are against an AtmB Protein and the amount of antibodies is greater than a standard determined from a sample from an uninfected individual.

A non-invasive method for detection, diagnosis or prediction of a GAS disease in a subject is provided and comprises: obtaining a sample of a biological fluid, in particular, blood, plasma, serum, urine or saliva, or a tissue sample from the subject; subjecting the sample to a procedure to detect GAS markers or antibodies against GAS markers in the blood, plasma, serum, urine, saliva or tissue; detecting, diagnosing, and predicting GAS disease by comparing the levels of GAS markers or antibodies to the levels of marker(s) or antibodies obtained from a control subject with no GAS disease.

In aspect, a method for monitoring the progression of a GAS disease in a patient the method is provided and comprises:
(a) detecting GAS markers or antibodies against GAS markers in a sample from the patient at a first time point;
(b) repeating step (a) at a subsequent point in time; and
(c) comparing the levels detected in (a) and (b), and therefrom monitoring the progression of the GAS disease.

A method is contemplated for determining the effect of an environmental factor on a GAS disease comprising comparing GAS markers in the presence and absence of the environmental factor.

Also provided is a method of assessing the efficacy of a therapy for inhibiting a GAS disease in a patient. A method therefore comprises comparing: (a) levels of GAS disease markers or antibodies against GAS markers in a first sample from the patient obtained from the patient prior to providing at least a portion of the therapy to the patient; and (b) levels of GAS disease markers or antibodies in a second sample obtained from the patient following therapy.

In an embodiment, a significant difference between the levels of GAS markers or antibodies in the second sample relative to the first sample is an indication that the therapy is efficacious for inhibiting GAS disease. In a particular embodiment, the method is used to assess the efficacy of a therapy for inhibiting GAS disease, where lower levels of GAS markers or antibodies in the second sample relative to the first sample, is an indication that the therapy is efficacious for inhibiting the disease. The “therapy” may be any therapy for treating GAS disease, including but not limited to antibiotics. Therefore, the method can be used to evaluate a patient before, during, and after therapy.

Certain methods employ binding agents (e.g. antibodies) that specifically recognize GAS markers. In an embodiment, methods are provided for determining the presence or absence of GAS disease in a patient, comprising the steps of (a) contacting a biological sample obtained from a patient with one or more binding agent that specifically binds to one or more GAS markers; and (b) detecting in the sample an amount of marker that binds to the binding agent, relative to a predetermined standard or cut-off value, and therefrom determining the presence or absence of GAS disease in the patient.

In another embodiment, a method is provided for diagnosing and monitoring a GAS disease in a subject by quantitating one or more GAS markers associated with the disease in a biological sample from the subject comprising (a) reacting the biological sample with one or more binding agent specific for the GAS markers (e.g. an antibody) that are directly or indirectly labelled with a detectable substance; and (b) detecting the detectable substance.

In another aspect a method is provided for using an antibody to detect expression of one or more GAS marker in a sample, the method comprising: (a) combining antibodies specific for one or more GAS marker with a sample under conditions which allow the formation of antibody:marker complexes; and (b) detecting complex formation, wherein complex formation indicates expression of the marker in the sample. Expression may be compared with standards and is diagnostic of a GAS disease.

Embodiments of the methods involve (a) reacting a biological sample from a subject with antibodies specific for one or more GAS markers which are directly or indirectly labelled with an enzyme; (b) adding a substrate for the enzyme wherein the substrate is selected so that the substrate, or a reaction product of the enzyme and substrate forms fluorescent complexes; (c) quantitating one or more GAS markers in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects without a GAS disease (e.g. uninfected individuals) wherein an increase in GAS marker levels compared with the control subjects is indicative of GAS disease.

In one embodiment, the method comprises the following steps:
(a) incubating a biological sample with first antibodies specific for one or more GAS markers which are directly or indirectly labeled with a detectable substance, and second antibodies specific for one or more GAS markers which are immobilized;
(b) detecting the detectable substance thereby quantitating GAS markers in the biological sample; and
(c) comparing the quantitated GAS markers with levels for a predetermined standard.

The standard may correspond to levels quantitated for samples from control subjects without a GAS disease (uninfected individuals) or from other samples of the subject. In an embodiment, increased levels of GAS markers as compared to the standard may be indicative of a GAS disease.

GAS marker levels can be determined by constructing an antibody microarray in which binding sites comprise immobilized antibodies (preferably monoclonal antibodies) specific to a substantial fraction of marker-derived GAS protein markers of interest.

Other methods employ one or more polynucleotides capable of hybridizing to one or more polynucleotides encoding GAS protein markers. Thus, methods for detecting GAS markers can be used to monitor a GAS disease by detecting GAS polynucleotide markers associated with the disease. Thus, a method is provided for diagnosing and monitoring a GAS disease in a sample from a subject comprising isolating nucleic acids, preferably mRNA, from the sample; and detecting GAS polynucleotide markers associated with the disease in the sample. The presence of different levels of GAS polynucleotide markers in the sample compared to a standard or control may be indicative of disease, disease stage, and/or a positive prognosis i.e. longer progression-free and overall survival.

Methods for determining the presence or absence of a GAS disease in a subject are also provided and comprise detecting in the sample levels of nucleic acids that hybridize to one or more GAS disease polynucleotide markers, comparing the levels with a predetermined standard or cut-off value, and therefrom determining the presence or absence of GAS disease in the subject. In an embodiment, methods are provided for determining the presence or absence of a GAS disease in a subject comprising (a) contacting a sample obtained from the subject with oligonucleotides that hybridize to one or more GAS disease polynucleotide markers; and (b) detecting in the sample a level of nucleic acids that hybridize to the polynucleotides relative to a predetermined cut-off value, and therefrom determining the presence or absence of GAS disease in the subject.

Within certain embodiments, the amount of polynucleotides that are mRNA are detected via polymerase chain reaction using, for example, oligonucleotide primers that hybridize to one or more GAS disease polynucleotide markers, or complements of such polynucleotides. Within other embodiments, the amount of mRNA is detected using a hybridization technique, employing oligonucleotide probes that hybridize to one or more GAS disease polynucleotide markers, or complements thereof.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more GAS polynucleotide markers in the sample. For mRNA the analyzing step may be accomplished using Northern Blot analysis to detect the presence of GAS polynucleotide markers. The analysis step may be further accomplished by quantitatively detecting the presence of GAS polynucleotide markers in the amplification product, and comparing the quantity of markers detected against a panel of expected values for the known presence of the markers in samples from uninfected individuals derived using similar primers.

Therefore, a method is provided wherein mRNA is detected by (a) isolating mRNA from a sample and combining the mRNA with reagents to convert it to cDNA; (b) treating the converted cDNA with amplification reaction reagents and nucleic acid primers that hybridize to one or more polynucleotides encoding GAS protein markers to produce amplification products; (d) analyzing the amplification products to detect an amount of mRNA encoding the GAS protein markers; and (e) comparing the amount of mRNA to an amount detected against a panel of expected values for normal samples (derived using similar nucleic acid primers).

In one embodiment, the methods described herein utilize the GAS polynucleotide markers placed on a microarray so that the expression status of each of the markers is assessed simultaneously.

In another aspect, a microarray is provided and comprises a defined set of genes (e.g., atmB, pbp1A, tdcF, coaA, Spy1674, Spy1784 and/or Spy1733). Further described is the use of the microarray as a prognostic tool to predict a GAS disease.

In an embodiment, oligonucleotide arrays are provided and comprise GAS marker sets described herein. The microarrays provided herein may comprise probes to markers able to distinguish a GAS disease. In one embodiment, oligonucleotide arrays are provided and comprise probes to a subset or subsets of gene markers up to a full set of markers which distinguish GAS disease.

Kits for carrying out the methods are also provided, and, in particular diagnostic methods. In an embodiment, a kit is for assessing whether a patient is afflicted with a GAS disease and it comprises reagents for assessing one or more GAS markers. Further provided are kits comprising marker sets described herein. In an aspect the kit contains a microarray ready for hybridization to target GAS markers, plus software for the data analyses.

Also provided is a diagnostic composition comprising one or more GAS marker. A composition is also provided comprising a probe that specifically hybridizes to a GAS marker or a fragment thereof, or an antibody specific for GAS markers or a fragment thereof. In another aspect, a composition is provided comprising one or more GAS polynucleotide marker specific primer pairs capable of amplifying the polynucleotides using polymerase chain reaction methodologies. The probes, primers or antibodies can be labeled with a detectable substance.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1. Growth curves of NZ131, GAS5448, and their corresponding atmB mutants in TH broth. Each curve represents an average of three independent experiments.

FIG. 2. Determination of the amount of cell-associated (A) and released (B) hyaluronic acid capsule from late-log phase cultures of NZ131, GAS5448 and their corresponding atmB mutants. Shown is the average of 3 independent cultures (±standard deviation) and statistical significance (P<0.05) between mutants and corresponding parent wild-type strains is indicated by the asterisk (*).

FIG. 3. Mean weight gain/loss (+range) of 5 mice each inoculated sub-cutaneously with NZ131, GAS5448, and their corresponding atmB mutants. Statistical significance (P<0.05) between mutants and corresponding parent wild-type strains is indicated by the asterisk (*).

FIG. 4. Average log₁₀ (CFU/mL) of bacteria recovered from the IP lavage (A & B) and spleen (C & D) at 12 and 24 h from mice infected intra-peritoneally with NZ131 (A & C), GAS5448 (B & D), and their respective atmB mutants.

### GLOSSARY

In accordance with the present invention there may be employed conventional biochemistry, enzymology, molecular biology, microbiology, and recombinant DNA techniques within the skill of the art. Such techniques are explained fully in the literature. See for example, Sambrook et al., Molecular Cloning: A Laboratory Manual, Third Edition (2001) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & Si. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

The terms “sample”, “biological sample”, and the like mean a material known or suspected of expressing or containing one or more GAS markers, especially AtmB Proteins. A test sample can be used directly as obtained from the source or following a pretreatment to modify the character of the sample. The sample can be derived from any biological source, such as tissues, extracts, or cell cultures, including cells, cell lysates, and physiological fluids, such as, for example, blood, plasma, serum, saliva, ocular lens fluid, cerebral spinal fluid, sweat, urine, milk, ascites fluid, synovial fluid, peritoneal fluid, lavage fluid, and the like. The sample can be obtained from animals, preferably mammals, most preferably humans. The sample can be treated prior to use, such as preparing plasma from blood, diluting viscous fluids, and the like. Methods of treatment of samples can involve filtration, distillation, extraction, concentration, inactivation of interfering components, the addition of reagents, and the like. In an embodiment the sample is a human physiological fluid. In a particular embodiment, the sample is human serum or urine.

The terms “subject”, “individual” or “patient” refer to a warm-blooded animal such as a mammal. In particular, the terms refer to a human. A subject, individual or patient may be afflicted with or suspected of having or being pre-disposed to a GAS disease. The term also includes domestic animals bred for food or as pets, including horses, cows, sheep, poultry, fish, pigs, cats, dogs, and zoo animals.

Methods herein for administering an agent or composition to subjects/individuals/patients contemplate treatment as well as prophylactic use. Typical subjects for treatment include persons susceptible to, suffering from or that have suffered a GAS disease.

The terms “peptide”, “polypeptide” and “protein” are used interchangeably and as used herein refer to more than one amino acid joined by a peptide bond.

“Optional” or “optionally” means that the subsequently described element, event or circumstance may or may not occur, and that the description includes instances where the element, event, or circumstance occurs and instances where it does not.

The term “effective amount” or “effective dose” refers to a non-toxic but sufficient amount of an agent (e.g. antibody) to provide the desired biological effect. The exact amount required will vary from subject to subject, depending on the species, age and general condition of the subject, the particular agent used, its mode of administration, and the like. An appropriate effective amount or effective dose may be determined by one of ordinary skill in the art using routine experimentation.

“Pharmaceutically acceptable” refers to a material that is not biologically or otherwise undesirable, i.e., the material may be administered to an individual without causing any undesirable biological effects or interacting in a deleterious manner with any of the other components of a composition in which it is contained.

“Synthetic” refers to items, e.g., peptides, which are not naturally occurring, in that they are isolated, synthesized or otherwise manipulated by man.

“Immunogenic” as used herein encompasses materials which are capable of producing an immune response.

“Composition” includes any composition of matter, including peptides, polypeptides, proteins, mixtures, vaccines, antibodies, or markers described herein.

A “GAS disease” means a disease associated with a group A Streptococcus, including without limitation, streptococcal sore throat (strep throat, pharyngitis), streptococcal skin infections (impetigo, cellulitis, erysipelas), scarlet fever, rheumatic fever, postpartum fever, wound infections, pneumonia, invasive group A strep infection, acute glomerulonephritis, necrotizing fasciitis and streptococcal toxic shock syndrome.

A “GAS marker” includes a polypeptide associated with GAS described herein (GAS protein marker), namely an AtmB Protein, and in some aspects, additionally a putative penicillin-binding protein 1a (pbp 1A) (Pbp1A Protein), a TdcF hypothetical protein (“Tdcf Protein”), CoaA pantothenate kinase (“CoA Protein”), Spy1674 (“Spy1674 Protein”), Spy1784 (“Spy1784 Protein”) and/or Spy1733 (“Spy1733 Protein”). A “GAS Marker” also includes a polynucleotide associated with GAS described herein (“GAS polynucleotide marker”), namely polynucleotides encoding an AtmB Protein (“atmB” or “AtmB polynucleotide”), and in some aspects, additionally polynucleotides encoding a putative penicillin-binding protein 1a (pbp 1A) (“pbp 1A” or “Pbp1A polynucleotide”), a TdcF hypothetical protein (“TdcF” or “tdcF polynucleotide”), CoaA Protein, Spy1674 Protein, Spy1784 Protein and/or Spy1733 Protein, especially a Pbp1A polynucleotide and/or a TdcF polynucleotide.

An “AtmB Protein” includes an AtmB polypeptide or protein of S. pyogenes and S. mutans, in particular the native-sequence polypeptide, isoforms, chimeric polypeptides, all homologs, fragments, precursors, complexes, and modified forms and derivatives thereof. Examples of amino acid sequences include the sequences of GeneID Nos. 1008547 and 1029136 and GenBank Accession Nos. NP 664037.1, AAM78840.1, NP 722245.1 and AAN59551.1 [SEQ ID NOs: 1, 2, 3 and 4].

A “Pbp1A Protein” includes a putative penicillin-binding protein 1a (pbp 1A) of S. pyogenes, in particular the native-sequence polypeptide, isoforms, chimeric polypeptides, all homologs, fragments, precursors, complexes, and modified forms and derivatives thereof. Examples of amino acid sequences include the sequences of GeneID No. 901903 and GenBank Accession Nos. NP 269695.1 and AAK34416.1 [SEQ ID NOs: 5 and 6].

A “TdcF Protein” includes a tdcF hypothetical protein of S. pyogenes, in particular the native-sequence polypeptide, isoforms, chimeric polypeptides, all homologs, fragments, precursors, complexes, and modified forms and derivatives thereof. Examples of amino acid sequences include the sequences of GeneID No. 993712 and GenBank Accession Nos. NP_{—}608077.1 spyM18 2121, and AAL98576.1 [SEQ ID NOs: 7 and 8].

A “CoaA Protein” includes a pantothenate kinase of S. pyogenes or S. agalactiae in particular the native-sequence polypeptide, isoforms, chimeric polypeptides, all homologs, fragments, precursors, complexes, and modified forms and derivatives thereof. Examples of amino acid sequences include the sequences of GeneID Nos. 4063674, 1013755 and 1029377 and GenBank Accession Nos. YP_{—}598668, NP_{—}721513.1, NP_{—}687963.1, AAN58819, and ABF34124 [SEQ ID NOs: 9, 10, 11, 12, and 13].

A “Spy1674 Protein” includes a putative ABC transporter (ATP-binding protein) of S. pyogenes, in particular the native-sequence polypeptide, isoforms, chimeric polypeptides, all homologs, fragments, precursors, complexes, and modified forms and derivatives thereof. Examples of amino acid sequences include the sequences of GeneID No. 901921 and GenBank Accession Nos. NP 269711.1 and NP 269711.1 [SEQ ID NO: 14].

A “Spy1784 Protein” includes a putative ABC transporter (ATP-binding protein) of S. pyogenes, in particular the native-sequence polypeptide, isoforms, chimeric polypeptides, all homologs, fragments, precursors, complexes, and modified forms and derivatives thereof. Examples of amino acid sequences include the sequences of GeneID No. 902015 and GenBank Accession Nos. NP 269798.1 and AAK34519 [SEQ ID NOs: 15 and 16].

A “Spy1733 Protein” includes a hypothetical protein Spy1733, amino acid permease, of S. pyogenes, in particular the native-sequence polypeptide, isoforms, chimeric polypeptides, all homologs, fragments, precursors, complexes, and modified forms and derivatives thereof. Examples of amino acid sequences include the sequences of GeneID Nos. 4064767, 4066845, and 2941009 and GenBank Accession Nos. YP_{—}601369, YP_{—}603268, YP_{—}061091, ABF36825, ABF38724, and AAT87908 [SEQ ID NOs: 17, 18, 19, 20, 21 and 22].

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 45%, 50%, 55%, 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Variants include, for instance, polypeptides wherein one or more amino acid residues are added to, or deleted from, the N- or C-terminus of the full-length or mature sequences of the polypeptide, including variants from other species, but excludes a native-sequence polypeptide. In one aspect, variants retain the immunogenic activity of the corresponding native-sequence polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); the BLASTP program, the BLASTN program, and the FASTA program (Altschul, S. F. et al. J. Molec. Biol. 215: 403-410, 1990). The BLASTX program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

A variant may be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which includes fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine polypeptide.

A “chimeric protein” or “fusion protein” comprises all or part (preferably biologically active) of a GAS protein marker operably linked to a heterologous polypeptide (i.e., a polypeptide other than a GAS protein marker). Within the fusion protein, the term “operably linked” is intended to indicate that a GAS protein marker and the heterologous polypeptide are fused in-frame to each other. The heterologous polypeptide can be fused to the N-terminus or C-terminus of a GAS protein marker. A useful fusion protein is a GST fusion protein in which a GAS protein marker is fused to the C-terminus of GST sequences. Chimeric and fusion proteins can be produced by standard recombinant DNA techniques.

A GAS protein marker may be prepared by recombinant or synthetic methods, or isolated from a variety of sources, or by any combination of these and similar techniques.

An “AtmB polynucleotide” or “atmB” includes polynucleotides that encode AtmB Proteins. Examples of nucleic acid sequences include the sequences of GeneID Nos. 1008547 and 1029136 and GenBank Accession Nos. AE014074.1, NP 722245.1 and AE014133.1.

A “Pbp1A polynucleotide” or “pbp1A” includes polynucleotides that encode Pbp1A Proteins. Examples of nucleic acid sequences include the sequences of GeneID No. 901903 and GenBank Accession Nos. NP 269695.1, AE006596.1 and AE004092.

A “TdcF polynucleotide” or “tdcF” includes polynucleotides that encode TdcF Proteins. Examples of nucleic acid sequences include the sequences of GeneID No. 993712 and GenBank Accession Nos. NP 608077.1, AAL98576.1 and AE10114.1.

A “CoaA polynucleotide” or “coaA” includes polynucleotides that encode CoaA Proteins. Examples of nucleic acid sequences include the sequences of GeneID Nos. 4063674 1013755, and 1029377 and GenBank Accession No. CP000260.

A “Spy1674 polynucleotide” or “Spy1674” includes polynucleotides that encode Spy1674 Proteins. Examples of nucleic acid sequences include the sequences of GeneID No. 901921 and GenBank Accession Nos. AE006597 and AE004092.

A “Spy1784 polynucleotide” or “Spy1784” includes polynucleotides that encode Spy1784 Proteins. Examples of nucleic acid sequences include the sequences of GeneID No. 902015 and GenBank Accession No. AE006605 and AE004092.

A “Spy1773 polynucleotide” or “Spy1773” includes polynucleotides that encode Spy1773 Proteins. Examples of nucleic acid sequences include the sequences of GeneID Nos. 4064767, 4066845, and 2941009 and GenBank Accession Nos. CP000261, CP000262, and CP000003.

The polynucleotide markers include complementary nucleic acid sequences, and nucleic acids that are substantially identical to these sequences (e.g. having at least about 45%, 50%, 55%, 60%, 65%, 70%, 75%, 80%, 85%, 90%, 95%, 97%, 98%, or 99% sequence identity).

Polynucleotide markers also include sequences that differ from a native sequence due to degeneracy in the genetic code. Polynucleotide markers also include nucleic acids that hybridize under stringent conditions, preferably high stringency conditions to a GAS polynucleotide marker. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

Polynucleotide markers also include truncated nucleic acids or nucleic acid fragments and variant forms of the nucleic acids that arise by alternative splicing of an mRNA corresponding to a DNA.

Polynucleotide markers are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotide markers for use in the methods may be of any length suitable for a particular method. In certain applications, the term refers to antisense polynucleotides (e.g. mRNA or DNA strand in the reverse orientation to sense cancer polynucleotide markers).

“Statistically different levels”, “significantly altered levels”, or “significant difference” in levels of markers in a patient sample compared to a control or standard (e.g. normal levels or levels in other samples from a patient) may represent levels that are higher or lower than the standard error of the detection assay. In particular embodiments, the levels may be 1.5, 2, 3, 4, 5, or 6 times higher or lower than the control or standard.

“Microarray” and “array,” refer to nucleic acid or nucleotide arrays or protein or peptide arrays that can be used to detect biomolecules associated with a GAS disease, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-bases long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include the LifeArray® software made by Incyte Genomics and the DermArray® software made by IntegriDerm (or Invitrogen). Pre-synthesized and amplified cDNA sequences are attached to the substrate of spotted arrays. Protein and peptide arrays also are known (see for example, Zhu et al., Science 293:2101 (2001).

“Binding agent” refers to a substance such as a polypeptide or antibody that specifically binds to one or more GAS markers. A substance “specifically binds” to one or more GAS markers if is reacts at a detectable level with one or more GAS markers, and does not react detectably with peptides containing an unrelated or different sequence. Binding properties may be assessed using an ELISA, which may be readily performed by those skilled in the art (see for example, Newton et al., Develop. Dynamics 197: 1-13, 1993).

A binding agent may be a ribosome, with or without a peptide component, an aptamer, an RNA molecule, or a polypeptide. A binding agent may be a polypeptide that comprises one or more GAS marker sequence, a peptide variant thereof, or a non-peptide mimetic of such a sequence.

An aptamer includes a DNA or RNA molecule that binds to nucleic acids and proteins. An aptamer that binds to a protein (or binding domain) of a GAS marker can be produced using conventional techniques, without undue experimentation. (For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)).

Antibodies for use herein include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al., U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant GAS markers may be utilized to prepare antibodies; (See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies). Antibodies specific for a GAS marker may also be obtained from scientific or commercial sources.

In an embodiment, antibodies are reactive against a GAS marker if they bind with a Kₐ of greater than or equal to 10⁻⁷ M.

### Markers

A set of markers correlated with GAS disease is provided herein. A set of these markers identified as useful for detection, diagnosis, prevention and therapy of GAS disease comprises an AtmB Protein or AtmB polynucleotide.

Gene marker sets that distinguish GAS disease and uses thereof are also provided. In an aspect, a method is provided for classifying a GAS disease comprising detecting a difference in the expression of a first plurality of genes relative to a control, the first plurality of genes consisting of AtmB polynucleotides, Pbp1A polynucleotides, and TdcF polynucleotides, and optionally CoaA polynucleotides, Spy1674 polynucleotides, Spy1784 polynucleotides and/or Spy1733 polynucleotides. In another specific aspect, the control comprises nucleic acids derived from a pool of samples from individual control patients.

Also provided is a marker set that distinguishes GAS disease and uses therefore comprising or consisting of one or more AtmB Proteins, Pbp1A Proteins, TdcF Proteins, AtmB polynucleotides, Pbp1A polynucleotides, and TdcF polynucleotides, and optionally CoaA Proteins, Spy1674 Proteins, Spy1784 Proteins, Spy1733 Proteins CoaA polynucleotides, Spy1674 polynucleotides, Spy1784 polynucleotides and/or Spy1733 polynucleotides.

In an aspect, a method is provided for classifying a GAS disease comprising detecting a difference in the expression of a first plurality of GAS markers relative to a control, the first plurality of GAS markers consisting of one or more AtmB Proteins, Pbp1A Proteins, TdcF Proteins, AtmB polynucleotides, Pbp1A polynucleotides, and TdcF Polynucleotides, and optionally CoaA polynucleotides, Spy1674 polynucleotides, Spy1784 polynucleotides and/or Spy1733 polynucleotides. In an aspect, the control comprises markers derived from a pool of samples from individual patients with no GAS disease.

Any of the markers provided herein may be used alone or with other markers of GAS disease, or with markers for other phenotypes or conditions.

### Nucleic Acid Methods/Assays

As noted herein a GAS disease may be detected based on the amount/level of GAS polynucleotide markers in a sample. Techniques for detecting polynucleotides such as polymerase chain reaction (PCR) and hybridization assays are well known in the art.

Probes may be used in hybridization techniques to detect GAS polynucleotide markers. The technique generally involves contacting and incubating nucleic acids (e.g. recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe, if any, are detected.

Nucleotide probes for use in the detection of nucleic acid sequences in samples may be constructed using conventional methods known in the art. Suitable probes may be based on nucleic acid sequences encoding at least 5 sequential amino acids from regions of a GAS polynucleotide marker, preferably they comprise 10-200, more particularly 10-30, 10-40, 20-50, 40-80, 50-150, 80-120 nucleotides in length.

The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone.

DNA can be obtained using standard methods such as polymerase chain reaction (PCR) amplification of genomic DNA or cloned sequences. (See, for example, in Innis et al., eds., 1990, PCR Protocols: A Guide to Methods and Applications, Academic Press Inc., San Diego, Calif.). Computer programs known in the art can be used to design primers with the required specificity and optimal amplification properties, such as the Oligo® version 5.0 software (National Biosciences). Controlled robotic systems may be useful for isolating and amplifying nucleic acids.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al., 1989, Molecular Cloning, A Laboratory Manual (2nd ed.). The nucleic acid probes may be used to detect GAS polynucleotide markers. The nucleotide probes may also be useful in the diagnosis of a GAS disease involving one or more GAS markers, in monitoring the progression of such disorder, or monitoring a therapeutic treatment.

The detection of GAS polynucleotide markers may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a polynucleotide encoding one or more GAS protein marker derived from a sample, wherein at least one of the oligonucleotide primers is specific for (i.e. hybridizes to) a polynucleotide encoding the GAS protein marker. The amplified cDNA is then separated and detected using techniques well known in the art, such as gel electrophoresis.

In order to maximize hybridization under assay conditions, primers and probes employed in the methods generally have at least about 60%, preferably at least about 75%, and more preferably at least about 90% identity to a portion of a polynucleotide encoding a GAS protein marker; that is, they are at least 10 nucleotides, and preferably at least 20 nucleotides in length. In an embodiment, the primers and probes are at least about 10-40 nucleotides in length.

Hybridization and amplification techniques described herein may be used to assay qualitative and quantitative aspects of GAS polynucleotide marker expression. For example, RNA may be isolated from a cell type or tissue known to express a GAS polynucleotide marker and tested utilizing the hybridization (e.g. standard Northern analyses) or PCR techniques referred to herein.

The primers and probes may be used in the above-described methods in situ, i.e., directly on tissue sections (fixed and/or frozen) of patient tissue obtained from biopsies or resections.

In an aspect, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a GAS marker sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected GAS disease and an individual who is not afflicted with a GAS disease. The reaction may be performed on several dilutions of cDNA spanning at least two orders of magnitude. A statistically significant difference in expression in several dilutions of the subject sample as compared to the same dilutions of the non-disease sample may be considered positive for the presence of a GAS disease.

In an embodiment, methods are described for determining the presence or absence of a GAS disease in a subject comprising (a) contacting a sample obtained from the subject with oligonucleotides that hybridize to GAS polynucleotide markers; and (b) detecting in the sample a level of nucleic acids that hybridize to the polynucleotides relative to a predetermined cut-off value, and therefrom determining the presence or absence of a GAS disease in the subject. In an aspect, the GAS polynucleotide markers are one or more of AtmB polynucleotides, Pbp1A polynucleotides, and TdcF Polynucleotides, and optionally CoaA polynucleotides, Spy1674 polynucleotides, Spy1784 polynucleotides and/or Spy1733 polynucleotides.

A method is provided wherein an GAS mRNA marker is detected by (a) isolating mRNA from a sample and combining the mRNA with reagents to convert it to cDNA; (b) treating the converted cDNA with amplification reaction reagents and nucleic acid primers that hybridize to one or more polynucleotides encoding GAS protein markers, to produce amplification products; (d) analyzing the amplification products to detect amounts of mRNA encoding GAS protein markers; and (e) comparing the amount of mRNA to an amount detected against a panel of expected values for normal subjects derived using similar nucleic acid primers.

GAS marker-positive samples or alternatively higher levels in patients compared to a control (e.g. non-infected individual) may be indicative of late stage disease, and/or that the patient is not responsive to therapy.

In another embodiment, methods are provided for determining the presence or absence of a GAS disease in a subject comprising (a) contacting a sample obtained from the subject with oligonucleotides that hybridize to one or more GAS polynucleotide markers; and (b) detecting in the sample levels of nucleic acids that hybridize to the polynucleotides relative to a predetermined cut-off value, and therefrom determining the presence or absence of a GAS disease in the subject.

In a further embodiment, a method is provided wherein atmB mRNA is detected by (a) isolating mRNA from a sample and combining the mRNA with reagents to convert it to cDNA; (b) treating the converted cDNA with amplification reaction reagents and nucleic acid primers that hybridize to atmB, to produce amplification products; (d) analyzing the amplification products to detect an amount of atmB mRNA; and (e) comparing the amount of mRNA to an amount detected against a panel of expected values for healthy individuals derived using similar nucleic acid primers.

Marker-positive samples or alternatively higher levels, in particular significantly higher levels of atmB in patients compared to a control (e.g. normal) are indicative of a GAS disease.

Oligonucleotides or longer fragments derived from GAS polynucleotide markers may be used as targets in a microarray. The microarray can be used to simultaneously monitor the expression levels of large numbers of genes and to identify genetic variants and mutations. The information from the microarray may be used to determine gene function, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), International Patent Publication No. WO95/251116; Shalon, D. et al. (I 995) International Patent Publication No. WO95/35505; Heller, R. A. et al. (1997) Proc. Natl. Acad. Sci. 94:2150-2155; and Heller, M. J. et al. (1997) U.S. Pat. No. 5,605,662).

Thus, an array is further provided and comprises one or more GAS polynucleotide markers and optionally other markers. The array can be used to assay expression of GAS polynucleotide markers in the array. The quantitation of expression of one or more GAS polynucleotide markers is permitted.

Microarrays typically contain at separate sites nanomolar quantities of individual genes, cDNAs, or ESTs on a substrate (e.g., nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Microarrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods can be immobilized to a solid support or surface which may be either porous or non-porous. For example, the probes can be attached to a nitrocellulose or nylon membrane or filter covalently at either the 3′ or the 5′ end of the polynucleotide probe. The solid support may be a glass or plastic surface. In an aspect, hybridization levels are measured to microarrays of probes consisting of a solid support on the surface of which are immobilized a population of polynucleotides, such as a population of DNA or DNA mimics, or, alternatively, a population of RNA or RNA mimics. A solid support may be a nonporous or, optionally, a porous material such as a gel.

A microarray is further provided and comprises a support or surface with an ordered array of hybridization sites or “probes” each representing one of the markers described herein. The microarrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Microarrays used herein are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In one aspect, the microarray is an array in which each position represents one of the GAS polynucleotide markers described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the GAS markers are present on the array.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. See, e.g., Egholm et al., 1993, Nature 363:566-568; U.S. Pat. No. 5,539,083.

Probes can be selected using an algorithm that takes into account binding energies, base composition, sequence complexity, cross-hybridization binding energies, and secondary structure (see Friend et al., International Patent Publication No. WO 01/05935, published Jan. 25, 2001).

Positive control probes, (e.g., probes known to be complementary and hybridize to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684). In an embodiment, microarrays are produced by synthesizing polynucleotide probes on a support wherein the nucleotide probes are attached to the support covalently at either the 3′ or the 5′ end of the polynucleotide.

Microarrays comprising a disclosed marker set are further provided. In one embodiment, a microarray is provided for distinguishing GAS disease samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the different nucleotide sequences selected from the group consisting of AtmB polynucleotides, Pbp1A polynucleotides, and TdcF polynucleotides, and optionally CoaA polynucleotides, Spy1674 polynucleotides, Spy1784 polynucleotides and/or Spy1733 polynucleotides.

### Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a GAS disease in a subject may be determined by (a) contacting a sample from the subject with a binding agent; (b) detecting in the sample a level of a GAS protein marker that binds to the binding agent; and (c) comparing the level of protein with a predetermined standard or cut-off value.

In one embodiment, the binding agent is an antibody. Antibodies specifically reactive with one or more GAS protein markers, or derivatives, such as enzyme conjugates or labeled derivatives, may be used to detect one or more GAS protein markers in various samples (e.g. biological materials). They may be used as diagnostic or prognostic reagents and they may be used to detect abnormalities in the levels of one or more GAS protein markers, and/or temporal, tissue, cellular, or subcellular location of one or more GAS protein markers. Antibodies may also be used to screen potentially therapeutic compounds in vitro to determine their effects on GAS diseases involving one or more GAS protein markers and other conditions. In vitro immunoassays may also be used to assess or monitor the efficacy of particular therapies.

In an aspect, a method is provided for monitoring or diagnosing a GAS disease in a subject by quantitating one or more GAS protein markers in a biological sample from the subject comprising reacting the sample with antibodies specific for one or more GAS protein markers, which are directly or indirectly labeled with detectable substances and detecting the detectable substances. In one embodiment, GAS protein markers are quantitated or measured.

In an aspect, a method for detecting a GAS disease is provided comprising:
(a) obtaining a sample suspected of containing one or more GAS protein markers associated with a GAS disease;
(b) contacting the sample with antibodies that specifically bind to the GAS protein markers under conditions effective to bind the antibodies and form complexes;
(c) measuring the amount of GAS protein markers present in the sample by quantitating the amount of the complexes; and
(d) comparing the amount of GAS protein markers present in the samples with the amount of GAS protein markers in a control, wherein a change or significant difference in the amount of GAS protein markers in the sample compared with the amount in the control is indicative of a GAS disease.

In an embodiment, a method for monitoring the progression of a GAS disease in an individual is provided and comprises:
(a) contacting antibodies which bind to one or more GAS protein markers with a sample from the individual so as to form complexes comprising the antibodies and one or more GAS protein markers in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the disease in the individual.

The amount of complexes may also be compared to a value representative of the amount of the complexes from an individual not afflicted with a GAS disease at different stages. A significant difference in complex formation may be indicative of advanced disease or an unfavorable prognosis.

In aspects for diagnosis and monitoring of a GAS disease, the GAS markers are one or more AtmB Proteins, Pbp1A Proteins, and TdcF Proteins, and optionally CoaA Proteins, Spy1674 Proteins, Spy1784 Proteins, and/or Spy1733 Proteins.

In embodiments of the methods described herein, an AtmB Protein is detected in samples and higher levels, in particular significantly higher levels compared to a control (normal or benign) is indicative of a GAS disease.

Antibodies may be used in any known immunoassays that rely on the binding interaction between antigenic determinants of one or more GAS protein marker and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more GAS protein marker in a sample may be accomplished by competitive or non-competitive immunoassay procedures in either a direct or indirect format. Detection of one or more GAS protein marker using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

According to one embodiment, an immunoassay for detecting one or more GAS protein markers in a biological sample comprises contacting binding agents that specifically bind to GAS protein markers in the sample under conditions that allow the formation of first complexes comprising a binding agent and GAS protein markers and determining the presence or amount of the complexes as a measure of the amount of GAS protein markers contained in the sample. In a particular embodiment, the binding agents are labeled differently or are capable of binding to different labels.

Antibodies may be used to detect and quantify one or more GAS protein markers in a sample in order to diagnose and treat a GAS disease. Immunohistochemical methods for the detection of antigens in tissue samples are well known in the art. For example, immunohistochemical methods are described in Taylor, Arch. Pathol. Lab. Med. 102:112 (1978). Briefly, a tissue sample obtained from a subject suspected of having a GAS disease is contacted with antibodies, preferably monoclonal antibodies recognizing one or more GAS protein markers. The site at which the antibodies are bound is determined by selective staining of the sample by standard immunohistochemical procedures. The same procedure may be repeated on the same sample using other antibodies that recognize one or more GAS protein markers. Alternatively, a sample may be contacted with antibodies against one or more GAS protein markers simultaneously, provided that the antibodies are labeled differently or are able to bind to a different label.

An antibody microarray in which binding sites comprise immobilized, preferably monoclonal, antibodies specific to a substantial fraction of GAS protein markers of interest can be utilized. Antibody arrays can be prepared using methods known in the art [see for example, Zhu et al., Science 293:2101 (2001) and reference 20].

Antibodies specific for one or more GAS protein markers may be labelled with a detectable substance and localised in biological samples based upon the presence of the detectable substance. Examples of detectable substances include, but are not limited to, the following: radioisotopes (e.g., ³H, ¹⁴C ³⁵S, ¹²⁵I, ¹³¹I) fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualized by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steroid isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as diethylene triamine pentaacetic acid (DTPA) or ethylenediaminetetraacetic acid (EDTA).

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more GAS protein markers. By way of example, if the antibody having specificity against one or more GAS protein markers is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications”, Anal. Biochem. 171:1-32, 1988 re methods for conjugating or labelling the antibodies with enzyme or ligand binding partner).

Cytochemical techniques known in the art for localizing antigens using light and electron microscopy may be used to detect one or more GAS protein markers. Generally, antibodies may be labeled with detectable substances and one or more GAS protein markers may be localised in tissues and cells based upon the presence of the detectable substances.

In the context of the methods described herein, the sample, binding agents (e.g. antibodies specific for one or more GAS protein markers), or one or more GAS protein markers may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a GAS marker may be immobilized using sheep anti-mouse IgG Fc fragment specific antibody coated on the carrier or support.

Where a radioactive label is used as a detectable substance, one or more GAS protein markers may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

In accordance with one embodiment, a method is provided wherein one or more GAS protein marker antibodies are directly or indirectly labelled with enzymes, substrates for the enzymes are added wherein the substrates are selected so that the substrates, or a reaction product of an enzyme and substrate, form fluorescent complexes with a lanthanide metal (e.g. europium, terbium, samarium, and dysprosium, preferably europium and terbium). A lanthanide metal is added and one or more GAS protein markers are quantitated in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,312,922 (Diamandis). Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed, in the method, may be 4-methylumbelliferyl phosphate, 5-fluorosalicyl phosphate, or diflunisal phosphate. The fluorescence intensity of the complexes is typically measured using a time-resolved fluorometer e.g. the CyberFluor® 615 Immunoanalyzer (Nordion. International, Kanata, Ontario).

One or more GAS protein marker antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for a GAS protein marker antibody is labeled with an enzyme.

In accordance with one embodiment, means are provided for determining one or more GAS protein markers in a sample by measuring one or more GAS protein markers by immunoassay. It will be evident to a skilled artisan that a variety of immunoassay methods can be used to measure one or more GAS protein markers. In general, an immunoassay method may be competitive or noncompetitive. Competitive methods typically employ an immobilized or immobilizable antibody to one or more GAS protein markers and a labeled form of one or more GAS protein markers. Sample GAS protein markers and labeled GAS protein markers compete for binding to antibodies to GAS protein markers. After separation of the resulting labeled GAS protein markers that have become bound to antibodies (bound fraction) from that which has remained unbound (unbound fraction), the amount of the label in either bound or unbound fraction is measured and may be correlated with the amount of to GAS protein markers in the test sample in any conventional manner, e.g., by comparison to a standard curve.

In an aspect, a non-competitive method is used for the determination of one or more GAS protein markers, with the most common method being the “sandwich” method. In this assay, two antibodies to GAS protein markers are employed. One of the antibodies to GAS protein markers is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises GAS protein markers bound by (“sandwiched” between) the capture and detection antibodies. In an embodiment, the label may be measured without separating the capture antibodies and liquid test mixture.

In a typical two-site immunometric assay for GAS protein markers, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. In a particular aspect, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

The above-described immunoassay methods and formats are intended to be exemplary and are not limiting.

### Antibody Methods

Methods and reagents are provided for detecting antibodies to GAS markers. Assays and devices for detecting antibodies are well known to persons skilled in the art. For example, an enzyme-linked immunosorbant assay (ELISA) may be used to detect antibodies. An ELISA assay can be performed by binding a GAS Marker (e.g. an AtmB Protein, and optionally one or more penicillin-binding proteins (PBPs) (e.g., pbp1A), TdcF Proteins, CoaA Proteins, Spy1674 Proteins, Spy1784 Proteins and Spy1733 Proteins, especially Pbp1A Proteins and/or TdcF Proteins) to a solid support, and then reacting the samples with the bound markers. A detectable substance can be used to detect bound antibody. For example, the detectable substance can be a labeled anti-human secondary antibody. In general, the method involves separating bound and free reagents using a series of dilution, incubation, and washing steps, and detecting the detectable substance which provides an indirect measure of the level of antibody in the samples. The ORIGEN electrochemiluminescence (ECL) detection system (Igen, Gaithersburg, Md.) can also be used to detect antibodies in a sample. Strepavidin-coated paramagnetic beads are used in the ECL system to detect immune complexes formed between antigen bound to a biotin-conjugated antibody. In addition, biosensor-based assays such as a BIACORE 2000 assay, a BIACORE X assay (the BIACORE 1000 assay, BIACORE 3000 assay, or BIACORE T100 assay) [Phamiacia (Uppsalla, Sweden)] or the IAsys™ biosensor [Fisons] may be used to detect antibodies in a sample. In one aspect, the amount of antibody that binds to a GAS Marker is directly proportional to the biosensor signal.

### Computer Systems

Analytic methods contemplated herein can be implemented by use of computer systems and methods described below and known in the art. Thus, computer readable media comprising one or more GAS markers, and optionally other markers are provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, computer readable medium having recorded thereon markers identified for patients and controls are contemplated.

“Recorded” refers to a process for storing information on computer readable medium. The skilled artisan can readily adopt any of the presently known methods for recording information on computer readable medium to generate manufactures comprising information on one or more GAS markers, and optionally other markers.

A variety of data processor programs and formats can be used to store information on one or more GAS markers and other markers on computer readable medium. For example, the information can be represented in a word processing text file, formatted in commercially-available software such as the WordPerfect® software and the Microsoft® Word software, or represented in the form of an ASCII file, stored in a database application, such as the DB2® databases, the Sybase® databases, the Oracle® databases, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

A medium is provided for holding instructions for performing a method for determining whether a patient has a GAS disease, comprising determining the presence or absence of one or more GAS markers, and optionally other markers, and based on the presence or absence of the one or more GAS markers and optionally other markers, determining a GAS disease, and optionally recommending a procedure or treatment.

In an aspect, a method is provided for detecting a GAS disease using a computer having a processor, memory, display, and input/output devices, the method comprising the steps of:
(a) creating records of one or more GAS markers, and optionally other markers of GAS disease, in a sample suspected of containing GAS markers;
(b) providing a database comprising records of data comprising one or more GAS markers, and optionally other markers; and
(c) using a code mechanism for applying queries based upon a desired selection criteria to the data file in the database to produce reports of records of step (a) which provide a match of the desired selection criteria of the database of step (b), the presence of a match being a positive indication that the markers of step (a) have been isolated from a sample of an individual with a GAS disease.

In an aspect, the computer systems, components, and methods described herein are used to monitor disease or determine the stage of disease.

### Kits

Also contemplated are kits for carrying out the methods described herein. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific GAS polynucleotide marker or antibody described herein, which may be conveniently used, e.g., in clinical settings to screen and diagnose patients and to screen and identify those individuals exhibiting a predisposition to developing a GAS disease.

In an embodiment, a container with a kit comprises a binding agent as described herein. By way of example, the kit may contain antibodies or antibody fragments which bind specifically to epitopes of one or more GAS protein markers and optionally other markers, antibodies against the antibodies labelled with an enzyme; and a substrate for the enzyme. The kit may also contain microtiter plate wells, standards, assay diluent, wash buffer, adhesive plate covers, and/or instructions for carrying out a method described herein using the kit.

In one aspect, the kit includes antibodies or fragments of antibodies which bind specifically to an epitope of one or more GAS protein markers and means for detecting binding of the antibodies to their epitopes, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use usually do not require other reagents, as, for example, where the kit contains a radiolabelled antibody preparation for in vivo imaging.

A kit may be designed to detect the level of GAS polynucleotide markers in a sample. In an embodiment, the polynucleotides encode one or more AtmB polynucleotides. Such kits generally comprise at least one oligonucleotide probe or primer, as described herein, that hybridizes to a polynucleotide encoding one or more GAS protein markers. Such an oligonucleotide may be used, for example, within a PCR or hybridization procedure. Additional components that may be present within the kits include a second oligonucleotide and/or a diagnostic reagent or container to facilitate detection of a polynucleotide encoding one or more GAS protein markers.

A kit is provided and contains a microarray described herein ready for hybridization to target GAS polynucleotide markers, plus software for the analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods described herein to evaluate compounds may be packaged into convenient kits described herein providing the necessary materials packaged into suitable containers.

A kit is also contemplated for assessing the presence of GAS, wherein the kit comprises antibodies specific for one or more GAS markers, or primers or probes for polynucleotides encoding same, and optionally probes, primers or antibodies specific for other markers associated with a GAS disease.

A kit for assessing the suitability of each of a plurality of test compounds for inhibiting a GAS disease in a patient is further provided. The kit comprises reagents for assessing one or more GAS markers, and optionally a plurality of test agents or compounds.

### Therapeutic Applications

One or more GAS markers may be targets for immunotherapy. Immunotherapeutic methods include the use of antibody therapy, in vivo vaccines, and ex vivo immunotherapy approaches.

In one aspect, one or more antibodies specific for one or more GAS protein markers, especially an AtmB Protein, are provided and may be used systemically to treat a GAS disease associated with the marker. In particular, the GAS disease is strep throat, scarlet fever, impetigo, cellulitis-erysipelas, rheumatic fever, acute glomerular nephritis, endocarditis, or necrotizing fasciitis and one or more GAS marker antibodies may be used systemically to treat such disease.

Thus, a method is provided for treating a patient susceptible to, or having a disease that expresses one or more GAS protein marker, especially AtmB Protein, comprising administering to the patient an effective amount of one or more antibody that binds specifically to one or more GAS protein marker.

One or more GAS marker antibodies may also be used in a method for selectively inhibiting the growth or, or killing GAS expressing one or more GAS marker comprising reacting one or more GAS marker antibody immunoconjugate or immunotoxin with the cell in an amount sufficient to inhibit the growth of, or kill GAS.

By way of example, unconjugated antibodies to GAS protein markers may be introduced into a patient such that the antibodies bind to GAS expressing GAS protein markers and mediate growth inhibition of such GAS (including the destruction thereof). In addition to unconjugated antibodies to GAS protein markers, one or more GAS protein marker antibodies conjugated to therapeutic agents (e.g. immunoconjugates) may also be used therapeutically to deliver the agent directly to one or more GAS expressing GAS protein markers and thereby destroy the GAS. Examples of such agents include abrin, ricin A, Pseudomonas exotoxin, or diphtheria toxin.

In the practice of a method described herein, GAS protein marker antibodies capable of inhibiting the growth of GAS expressing GAS protein markers are administered in a therapeutically effective amount to patients with a GAS disease. A specific and effective treatment for a GAS disease may therefore be provided. The antibody therapy methods described herein may be combined with other therapies including antibiotics.

GAS protein marker antibodies useful in treating a GAS disease include those that are capable of initiating a potent immune response against the disease and those that are capable of direct cytotoxicity. In this regard, GAS protein marker antibodies may elicit cell lysis by either complement-mediated or antibody-dependent cell cytotoxicity (ADCC) mechanisms, both of which require an intact Fc portion of the immunoglobulin molecule for interaction with effector cell Fc receptor sites or complement proteins.

GAS protein marker antibodies that exert a direct biological effect on GAS may also be useful herein. Such antibodies may not require the complete immunoglobulin to exert the effect. Potential mechanisms by which such directly cytotoxic antibodies may act include inhibition of cell growth. The mechanism by which a particular antibody exerts an effect may be evaluated using any number of in vitro assays designed to determine ADCC, antibody-dependent macrophage-mediated cytotoxicity (ADMMC), complement-mediated cell lysis, and others known in the art.

The methods contemplate the administration of single GAS marker antibodies (e.g., AtmB Protein) as well as combinations, or “cocktails”, of different individual antibodies such as those recognizing different epitopes of other markers (e.g., Pbp1A Protein or TdcF Protein). Such cocktails may have certain advantages inasmuch as they contain antibodies that bind to different epitopes of GAS markers. Such antibodies in combination may exhibit synergistic therapeutic effects. In addition, the administration of one or more GAS protein marker specific antibodies may be combined with other therapeutic agents, including but not limited to antibiotics. The GAS marker specific antibodies may be administered in their “naked” or unconjugated form, or may have therapeutic agents conjugated to them.

The GAS protein marker specific antibodies used herein may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16. sup.th Edition, A. Osal., Ed., 1980).

One or more GAS protein marker specific antibody formulations may be administered via any route capable of delivering the antibodies to the disease site. Routes of administration include, but are not limited to, intravenous, intraperitoneal, intramuscular, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of disease and the severity, stage of the disease, the binding affinity and half life of the antibodies used, the degree of GAS marker expression in the patient, the extent of GAS markers, the desired steady-state antibody concentration level, frequency of treatment, and the influence of any therapeutic agents used in combination with the treatment method. Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve disease inhibition or regression. Direct administration of one or more GAS marker antibodies is also possible and may have advantages in certain situations.

Patients may be evaluated for serum GAS markers in order to assist in the determination of the most effective dosing regimen and related factors. Assay methods described herein, or similar assays, may be used for quantitating circulating GAS marker levels in patients prior to treatment. Such assays may also be used for monitoring throughout therapy, and may be useful to gauge therapeutic success in combination with evaluating other parameters such as serum levels of GAS markers.

Further provided are vaccines formulated to contain one or more GAS marker or fragment thereof. In an embodiment, a method is provided for vaccinating an individual against one or more GAS protein marker, especially AtmB Protein, comprising the step of inoculating the individual with the marker or fragment thereof that lacks activity, wherein the inoculation elicits an immune response in the individual thereby vaccinating the individual against the marker.

Viral gene delivery systems may be used to deliver one or more GAS polynucleotide markers. Various viral gene delivery systems which can be used in the practice of this aspect include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding one or more GAS protein marker or fragment thereof introduced into the patient (e.g., intramuscularly) to induce a response.

Anti-idiotypic GAS protein marker specific antibodies can also be used in therapy as a vaccine for inducing an immune response to GAS that express one or more GAS markers. The generation of anti-idiotypic antibodies is well known in the art and can readily be adapted to generate anti-idiotypic GAS protein marker specific antibodies that mimic an epitope on one or more GAS protein markers (see, for example, Wagner et al., 1997, Hybridoma 16: 33-40; Foon et al., 1995, J Clin Invest 96: 334-342). Such an antibody can be used in anti-idiotypic therapy as presently practiced with other anti-idiotypic antibodies directed against antigens associated with disease.

Genetic immunization methods may be utilized to generate prophylactic or therapeutic humoral and cellular immune responses directed against GAS expressing one or more GAS protein markers. One or more DNA molecules encoding GAS markers, constructs comprising DNA encoding one or more GAS markers/immunogens and appropriate regulatory sequences may be injected directly into muscle or skin of an individual, such that the cells of the muscle or skin take-up the construct and express the encoded GAS markers/immunogens. The GAS markers/immunogens may be expressed as cell surface proteins or be secreted. Expression of one or more GAS markers results in the generation of prophylactic or therapeutic humoral and cellular immunity against a GAS disease. Various prophylactic and therapeutic genetic immunization techniques known in the art may be used.

In another aspect, methods are provided for selectively inhibiting GAS expressing GAS protein markers by reacting any one or a combination of the immunoconjugates with a GAS in an amount sufficient to inhibit the GAS. Amounts include those that are sufficient to kill the GAS or sufficient to inhibit cell growth

Vectors derived from retroviruses, adenovirus, herpes or vaccinia viruses, or from various bacterial plasmids, may be used to deliver polynucleotides encoding GAS protein markers to a targeted site. Methods well known to those skilled in the art may be used to construct recombinant vectors that will express antisense polynucleotides for GAS protein markers. (See, for example, the techniques described in Sambrook et al. (supra) and Ausubel et al. (supra)).

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome is well known in the art.

One or more GAS markers and fragments thereof may be used in the treatment of a GAS disease in a subject. The GAS markers may be formulated into compositions for administration to subjects suffering from a GAS disease. Therefore, a composition is provided and comprises one or more GAS markers, or a fragment thereof, and a pharmaceutically acceptable carrier, excipient or diluent. A method for treating or preventing a GAS disease in a subject is also provided comprising administering to a patient in need thereof, one or more GAS markers, or a composition described herein.

Further provided is a method of inhibiting a GAS disease in a patient comprising:
(a) obtaining a sample containing GAS markers from the patient;
(b) separately maintaining aliquots of the sample in the presence of a plurality of test agents;
(c) comparing levels of one or more GAS markers in each aliquot;
(d) administering to the patient at least one of the test agents which alters the levels of the GAS markers in the aliquot containing that test agent, relative to the other test agents.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington: The Science and Practice of Pharmacy (21^{St} Edition. 2005, University of the Sciences in Philadelphia (Editor), Mack Publishing Company), and in The United States Pharmacopeia: The National Formulary (USP 24 NF19) published in 1999). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment, including without limitation antibiotics. The compositions described herein may be administered concurrently, separately, or sequentially with other therapeutic agents or therapies.

The therapeutic activity of compositions and agents/compounds identified using a method herein may be evaluated in vitro and in vivo using suitable assays and animal models, including the assays and models described in the example.

The following non-limiting examples are illustrative of the present invention:

### Example 1

Experimental Procedures

Bacterial strains, media, and growth conditions: Two strains of S. pyogenes, GAS5448 (M1 serotype) and NZ131 (M49 serotype), and derivates thereof were used in this study. The streptococci were grown in Todd Hewitt (TH) broth (Difco Laboratories, Michigan) or Columbia Blood Agar (CBA) plates (Oxoid, Ontario), incubated at 37° C. microaerobically with 5% CO₂. For growth of the atmB in-frame allelic replacement mutant strains, erythromycin (erm) was added to the medium at a concentration of 2.0 μg/mL. Inocula for the murine infection studies were prepared from overnight cultures of GAS sub-cultured into 20 mL of TH broth and grown at 37° C. microaerobically with 5% CO₂ until the culture reached mid-logarithmic growth phase (O.D._{600 nm}=0.4). Subsequently, the culture was centrifuged at 39,410×g at 4° C. for 10 min, the pellet washed once in 1× phosphate-buffered saline (PBS), and re-suspended in 1×PBS to the desired inoculum.

Construction of atmB in-frame allelic replacement mutants: Insertional inactivation of the atmB gene in GAS5448 and NZ131 wild-type strains has been described previously (Salim, K. Y., Cvitkovitch, D. G. et al., Infect Immun. 2005, 73(9): 6026-6038). Selection on these single-crossover Campbell-type genomic insertional mutants was relaxed by serial passage at 30° C. without antibiotics to promote a double cross-over event. In-frame allelic replacement mutants were identified as chloramphenicol susceptible (Cm^{S}) and erythromycin resistant (Erm^{R}) when grown at 37° C. in broth. Genomic DNA of the Cm^{S} Erm^{R} isolates was examined for erm and atmB by PCR and Southern analysis to confirm the mutants. The resultant atmB in-frame allelic replacement mutants in GAS5448 and NZ131 strains were termed GAS5448:atmBΔerm and NZ131:atmBΔerm, respectively.

Growth rate comparison: The growth kinetics of the wild-type strains and their corresponding atmB mutants were analyzed using a Bioscreen® microbiology reader (Bioscreen C Labsystems, Helsinki, Finland). Overnight cultures of each strain in TH broth were sub-cultured in triplicate into microtiter plate wells containing 300 μL of TH medium. Bioscreen parameters included growth at 37° C. for 16 h and O.D.₆₀₀ was recorded every 20 min.

Whole blood killing assay: The Lancefield bactericidal assay for GAS was modified to examine whole blood killing of GAS in human blood (Lancefield, R. C. J. Exp Med. 1957 Oct. 1; 106(4):525-44). Briefly, the GAS strains were grown to mid-log phase (optical density at 600 nm [O.D.₆₀₀ₘ]=0.4), washed and re-suspended in 1×PBS, and 10² CFU inoculated into 600 μl of fresh human blood. This mixture was incubated at 37° C. for 2 h with orbital shaking, and plated on CBA plates for enumeration of surviving bacteria. The experiment was conducted with blood samples from 8 healthy volunteers.

Hyaluronic acid quantitation: The amount of hyaluronic acid capsule produced by is each GAS strain was determined as described previously (Schrager, H. M., Rheinwald, J. G. et al., J. Clin. Invest 1996, 98(9): 1954-8). Briefly, cells from a 10-mL late log-phase culture were washed 2× with distilled water, then re-suspended in 0.5 mL distilled water. Capsule was released by shaking with 1 mL chloroform and centrifuged. A 50 μL-aliquot of the aqueous phase containing hyaluronic acid content was added to 2 mL of a solution containing 20 mg of 1-ethyl-2-[3-(1-ethylnaphtho-[1,2-d]thiazolin-2-ylidene)-2-methylpropenyl]naptho-1,2-d] thiazolium bromide (Stains All, Sigma) and 60 μL of glacial acetic acid in 100 mL of 50% formamide. The absorbance at 640 nm determined and compared with a standard curve generated using known concentrations of hyaluronic acid (Fluka, Switzerland). To determine the amount of hyaluronic acid released into the supernatant, the GAS strains grown as described above were removed by centrifugation at 2,000×g for 10 min, the supernatant filter-sterilized, adjusted to a final concentration of 0.5% hexadecylpyridium chloride (Sigma-Aldrich, Missouri) and incubated for 1 hr at 37° C. as described previously (Leonard, B. A., Woischnik, M. et al., Infect Immun 1998 66(8): 3841-7). This mixture centrifuged at 4,000×g for 30 min and the pellet re-dissolved in 2 mL of 0.5 M NaCl and 3 volumes of ethanol, and incubated overnight at −20° C. The hyaluronic acid precipitant was collected by centrifugation at 4,000×g for 30 min, dissolved in distilled water, and measured as described above. Quantitation of capsule per strain was performed in 3 independent experiments.

Dermonecrotic murine model: Four-week-old, immunocompetent, female, cr1:SKH1 (hrhr) hairless mice (Charles River Laboratories, Massachusetts) were inoculated sub-cutaneously with either wild-type or mutant strains of GAS (Betschel, S. D., Borgia, S. M. et al., Infect Immun. 66:1671-1679). Briefly, bacteria in 1×PBS were mixed 1:1 with 20 μg/mL Cytodex beads (Sigma-Aldrich) to the desired inoculum of 10⁶ colony-forming units (CFU). Groups of 5 mice were inoculated per strain and monitored for lesion formation and weight loss for 72 h at which time the mice were sacrificed, lesions photographed with a Powershot G6 camera (Canon, Mississauga) and images analyzed with the Openlab™ 4.0 imaging program (Improvision, England) to obtain lesion surface area. The lesions were then excised, ground in a disposable tissue grinder (Kendall, Mass.) and serially diluted in sterile 1×PBS to determine CFU/mL of bacteria. All experimental procedures were conducted in accordance with the principles of the Animal Care Committee of Mount Sinai Hospital, Toronto, Canada.

Septicemic murine model: Four- to six-week old immunocompetent, female, CD-1 mice (Charles River Laboratories) were inoculated intra-peritoneally as described previously (Gryllos, I., Cywes, C. et al., Mol Microbiol 2001, 42(1) 61-74). Groups of 20 mice each were intra-peritoneally inoculated with each GAS strain at a dose of 10⁸ CFU. Groups of 5 mice per group were sacrificed at 12 h and 24 h post-inoculation, blood samples collected via cardiac puncture, spleens homogenized in 1 mL 1× PBS, and intra-peritoneal (IP) lavage performed with 1 mL 1×PBS. The bacteria in the blood, spleen, and IP lavage were enumerated by plating on CBA plates. The remaining 10 mice per group were monitored daily for up to 13 days as part of a survival assay. All experimental procedures were conducted in accordance with the principles of the Animal Care Committee of Mount Sinai Hospital, Toronto, Canada.

Statistical analyses: The data was compared utilizing an Unpaired Student's t-test and a P value<0.05 was considered significant.

### Results

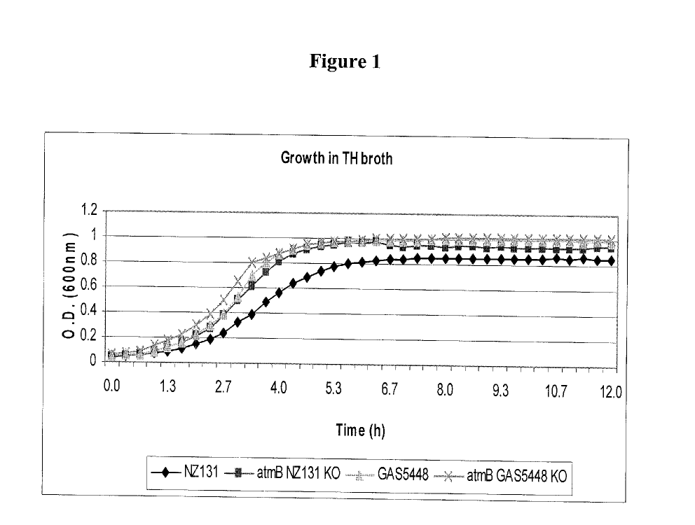

Confirmation of atmB mutants and growth rate comparisons: To characterize the role of atmB in GAS virulence, in-frame allelic replacement mutants were constructed in two M serotype strains of GAS. These mutants were confirmed by PCR and Southern analysis. To ensure that attenuation of virulence of the mutants was not a result of reduced growth, the growth rates of both the GAS wild-type strains and their corresponding atmB mutants were analyzed. The growth rates of the mutants and wild-type strains were shown to be equivalent (FIG. 1).

Survival in whole blood: A whole blood killing assay was conducted on the GAS strains to determine whether the loss of atmB reduces the ability of GAS to survive in human blood. Both the wild-types strains not only survived but replicated in ⅞ blood samples, whereas the NZ131:atmBΔerm mutant was cleared in all 8 samples and the GAS5448:atmBΔerm mutant survived in only ⅛ blood samples (Table 1). Thus, the mutants were significantly attenuated in their capacity to replicate in human blood.

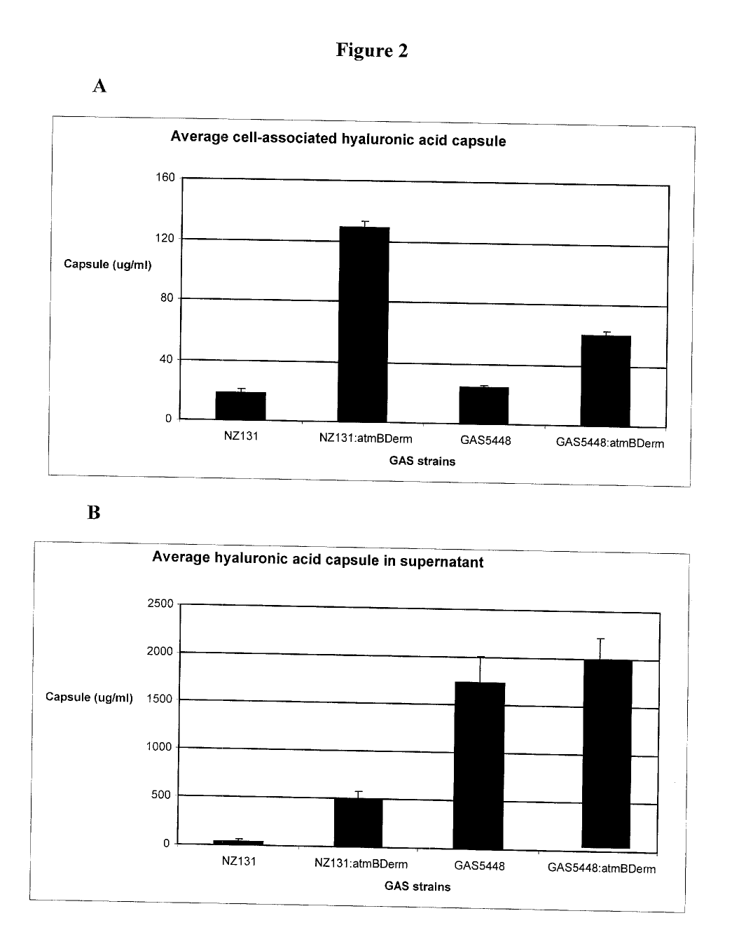

Hyaluronic acid content: The hyaluronic acid capsule that was released into the supernatant and associated with the cell surface was determined in both the mutants and wild-type GAS strains. Significantly more hyaluronic acid capsule was produced by the NZ131:atmBΔerm mutant relative to its wild-type parent strain both in the supernatant and on the cell surface (FIG. 2). Whereas a comparison between the GAS5448:atmBΔerm mutant and its corresponding parental wild-type strain indicated that the mutant produced significantly more cell-associated capsule, however, there was no significant difference in the amount of hyaluronic acid capsule released into the supernatant (FIG. 2).

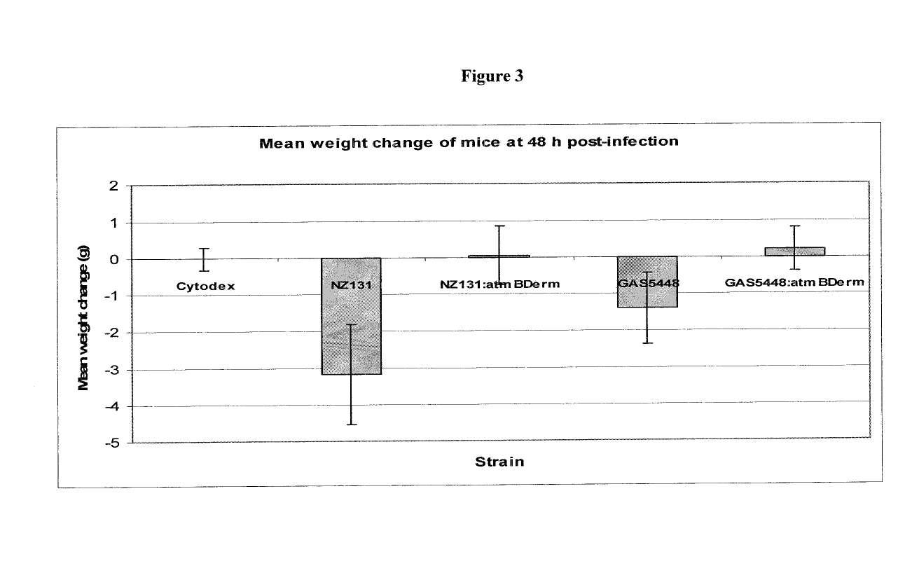

Attenuation of virulence of atmB mutants in the dermonecrotic murine model: To determine the effect of the loss of atmB on the virulence of GAS, the sub-cutaneous murine model of invasive GAS disease was utilized and mice were monitored for lesion formation and weight loss/gain, and the bacteria were enumerated at the infection site. None of the mice infected with NZ131:atmBΔerm formed a lesion and only ⅕ mice inoculated with the GAS5448:atmBΔerm mutant formed a lesion (Table 2). By contrast, almost all the mice (⅘ each) infected with NZ131 and GAS5448 wild-type strains formed lesions (Table 2). Furthermore, ⅖ mice infected with NZ131:atmBΔerm and ⅗ mice infected with GAS5448:atmBΔerm had cleared the bacteria from the inoculation site, whereas none of the mice infected with the wild-type strains were able to eliminate the bacteria from the inoculation site (Table 2). Finally, there was a significant weight loss in the mice inoculated with the wild-type strains relative to those inoculated with their respective atmB mutants at 48 h post-infection (FIG. 3).

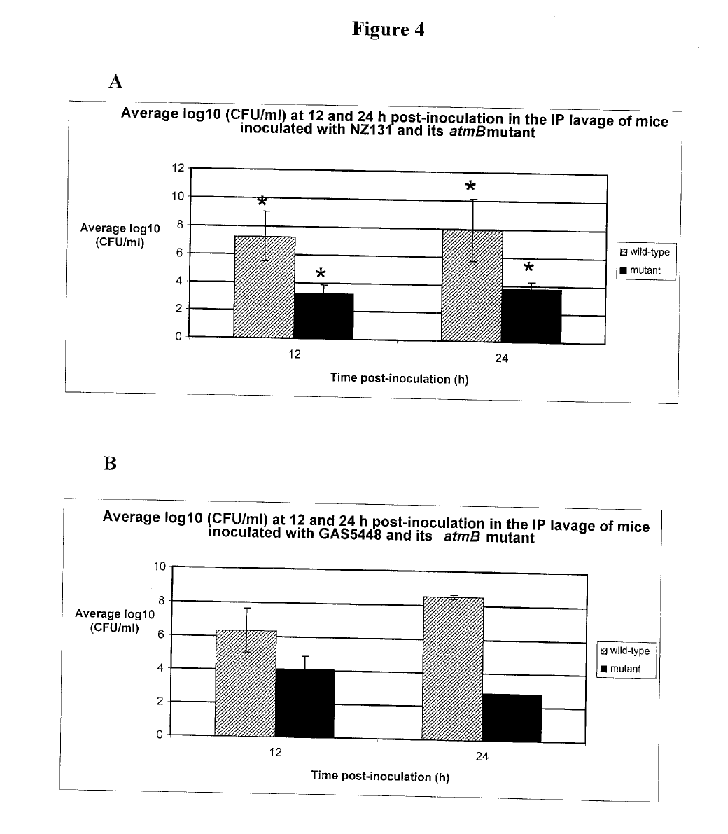

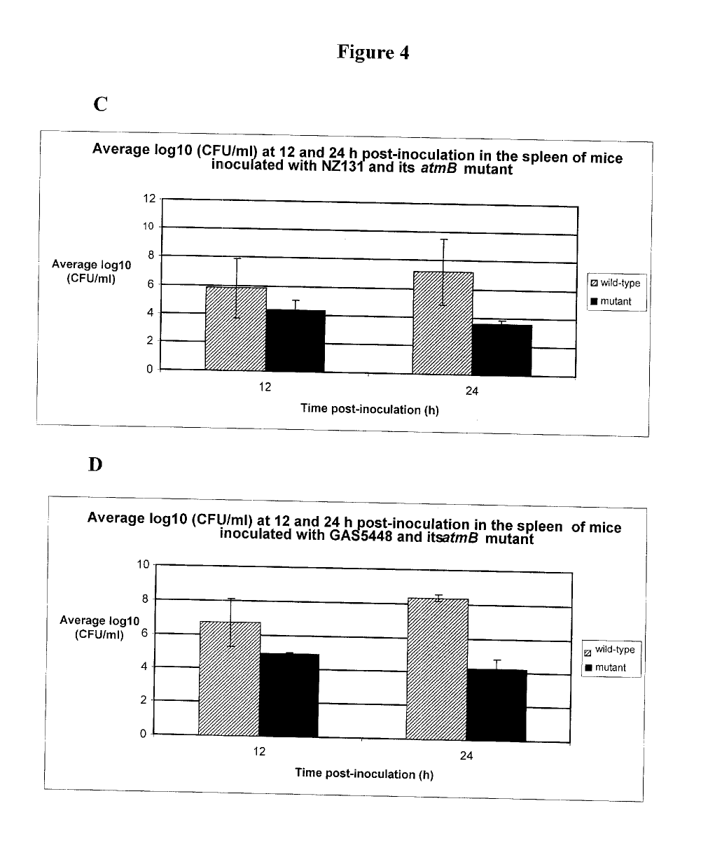

Attenuation of virulence of atmB mutants in the septicemic murine model: The atmB mutants were also tested relative to the wild-type strains in the septicemic murine model of GAS disease via intra-peritoneal inoculation. The survival of mice recorded over a period of 13 days showed that all the mice inoculated with the parent wild-type strains died within 24 h, whereas all the mice inoculated with NZ131:atmBΔerm survived for the entire duration of the assay and 70% of the mice inoculated with GAS5448:atmBΔerm survived for up to 13 days (Table 3). The eradication of the atmB mutants from the IP lavage was evident within 24 h post-inoculation and by 13 days the mutants were completely cleared from the IP cavity (Table 3). In fact, there was a statistically significant difference between the log₁₀ (CFU/mL) of the bacteria recovered from the IP lavage even at 12 h post-inoculation between the atmB mutants and their respective wild-type strains (FIGS. 4A & B). Although none of the mice infected with either the mutants or the wild-type GAS strains were able to clear the bacteria from the spleen at 24 h post-inoculation there was a statistically significant difference in the log₁₀ (CFU/mL) between each mutant and its corresponding wild-type strain (FIGS. 4C & D).

### Discussion

The aim of this study was to investigate the role of atmB in the pathogenesis of S. pyogenes invasive infections. This gene was first identified as an in vivo induced antigen utilizing In Vivo Induced Antigen Technology (the IVIAT technology) (Salim, K. Y., Cvitkovitch, D. G. et al., Infect Immun 2005, 73(9):6026-6038). This technology allowed for the identification of antigenic determinants expressed during disease with convalescent sera from patients with invasive disease. Insertionally inactivated mutants of atmB in two M serotype strains (M1 and M49) were analyzed in a murine subcutaneous model of S. pyogenes invasive disease. Both mutants were significantly attenuated in their capacity to cause lesions in mice relative to the wild-type parent strains (Salim, K. Y., Cvitkovitch, D. G. et al., supra). To ensure that the effect on virulence was indeed a result of the loss of atmB not due to downstream polar effects of the insertional inactivation in-frame allelic replacement mutants of atmB were constructed with the same M serotype strains of S. pyogenes (M1 and M49).

The in-frame allelic replacement mutants of atmB constructed in GAS5448 (M1 serotype) and NZ131 (M49 serotype) strains utilizing a temperature-sensitive pVE6007 were confirmed utilizing PCR and Southern analysis (data not shown). The growth rates of the mutants and the parent strains was equivalent thus indicating that any attenuation of virulence of the atmB mutants is not a result of reduced growth (FIG. 1). The up-regulation of atmB in S. pyogenes during interaction with human polymorphonuclear leukocytes suggested a possible role for atmB in S. pyogenes evasion of phagocytosis. Hence, the atmB mutants were tested utilizing a whole blood killing assay.

A characteristic feature of virulent S. pyogenes is its ability not only to survive but also replicate in human blood in the absence of opsonizing antibodies (Lancefield, R. C). This feature is attributed to the organism's capacity to evade phagocytosis. A number of virulence factors have been shown to contribute to phagocytic resistance in S. pyogenes. These include M proteins, hyaluronic acid capsule, C5a peptidase, streptococcal inhibitor of complement, and streptococcal pyrogenic exotoxins (Dale, J. B., Washburn, R. G. et al., Infect Immun 1996 64(5):1495-501; Eriksson, A. and Norgren, M. FEMS Immunol Med. Microbiol. 1999, 25(4): 355-63; Fernie-King, B. A., Seilly, D. J. et al., Infect Immun. 2002 70(9): 4908-16; Ji, Y., McLandsborough, L. et al., Infect Immun 1996, 64(2): 503-10; Wessels, M. R., Moses, A. E. et al., Proc Natl Acad Sci USA 1991 88(19): 8317-21). In addition to the aforementioned virulence factors, atmB also appears to be involved in the evasion of phagocytosis by S. pyogenes. In the whole blood killing assay the atmB mutants were significantly attenuated relative to their respective parent wild-type strains. The NZ131:atmBΔerm mutant was eradicated in all 8 blood samples tested and the GAS5448:atmBΔerm mutant survived in only ⅛ of the samples (Table 1). Conversely, both wild-type strains not only survived but replicated in ⅞ of the blood samples tested (Table 1). The atmB gene could either directly or indirectly or through the interaction with other virulence factors facilitate survival in blood. Undoubtedly the antiphagocytic property of GAS is complex. However, the experiments in this study clearly indicate that in the absence of atmB the potential for GAS to evade killing is abrogated.

TABLE 1
The survival and average log₁₀ (CFU/mL) of NZ131,
GAS5448 and their corresponding atmB mutants following
growth in fresh human blood at 37° C. for 2 h.

| | | AVERAGE LOG₁₀ |
|---|---|---|
| | | (CFU/ML) |
| | % SURVIVAL | (±STANDARD |
| STRAIN | IN BLOOD | DEVIATION) |
| NZ131 | 88% (7/8) | 3.80 (±0.62) |
| NZ131:atmBΔerm | 0% (0/8) | 0.00 (±0.00) |
| GAS5448 | 88% (7/8) | 4.40 (±0.78) |
| GAS5448:atmBΔerm | 13% (1/8) | 2.68 (±0.00) |

TABLE 2
Results of 5 mice each inoculated sub-cutaneously with NZ131, GAS5448, and their corresponding atmB
mutants at 72 h post-infection. Recorded below are the % of mice with lesions, average lesion surface
area, % eradication of bacteria, and enumeration of bacteria at the inoculation site.

| | | AVERAGE | | | | |
|---|---|---|---|---|---|---|
| | | SURFACE AREA | | | | AVERAGE LOG₁₀ |
| | | OF LESIONS (MM²) | | | | (CFU/ML) |
| | % OF MICE | (±STANDARD | | % ERADICATION | | (±STANDARD |
| STRAIN | WITH LESIONS | DEVIATION) | | OF BACTERIA | | DEVIATION) |
| NZ131 | 80% (4/5) | 24.50 | (±15.8) | 0% | (0/5) | 8.12 (±0.6) |
| NZ131:atmBΔerm | 0% (0/5) | 0 | (±0.0) | 40% | (2/5) | 4.18 (±1.6) |
| GAS5448 | 80% (4/5) | 13.55 | (±10.3) | 0% | (0/5) | 7.77 (±0.4) |
| GAS5448:atmBΔerm | 20% (1/5) | 4.48 | (±0.0) | 60% | (3/5) | 5.67 (±2.0) |

TABLE 3
Survival assay and bacterial enumeration in the spleen and IP lavage of mice inoculated
intra-peritoneally with NZ131, GAS5448, and their corresponding atmB mutants.

| | TIME POST- | | | % ERADICATION | | % ERADICATION | |
|---|---|---|---|---|---|---|---|
| | INFECTION | % SURVIVAL | | OF BACTERIA | | OF BACTERIA | |
| STRAIN | (DAYS) | OF MICE | | FROM SPLEEN | | FROM IP LAVAGE | |
| NZ131 | 1 | 0% | (0/10) | 0% | (0/10) | 0% | (0/10) |
| NZ131:atmBΔerm | 1 | 100% | (10/10) | 0% | (0/5) | 20% | (1/5) |
| GAS5448 | 1 | 0% | (0/10) | 0% | (0/10) | 0% | (0/10) |
| GAS5448:atmBΔerm | 1 | 100% | (10/10) | 0% | (0/5) | 80% | (4/5) |
| NZ131 | 13 | 0% | (0/10) | n/a | | n/a | |
| NZ131:atmBΔerm | 13 | 100% | (10/10) | 100% | (10/10) | 100% | (10/10) |
| GAS5448 | 13 | 0% | (0/10) | n/a | | n/a | |
| GAS5448:atmBΔerm | 13 | 70% | (7/10) | 71% | (5/7) | 100% | (7/7) |

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the antibodies, methodologies etc. which are reported therein which might be used in connection with the invention. Nothing herein is to be construed as an admission that the invention is not entitled to antedate such disclosure by virtue of prior invention.

We claim:

1. An immunogenic composition for protecting mammals against infection by group A Streptococcus (GAS) comprising an effective amount of a region of a group A Streptococcus atmB putative lipoprotein.

2. An immunogenic composition as claimed in claim 1, wherein the region of a group A Streptococcus atmB putative lipoprotein defines an epitope which induces the formation of bactericidal antibodies against GAS.

3. A composition as claimed in claim 1, wherein the region of a group A Streptococcal atmB putative lipoprotein is immunoreactive and found in the most prevalent GAS serotypes associated with a GAS disease.

4. An immunogenic composition as claimed in claim 1, comprising a synthetic peptide about 5 to 200 amino acids in length which are portions of a group A Streptococcus atmB putative lipoprotein.

5. An immunogenic composition as claimed in claim 4, wherein the synthetic peptide is about 10 to 150 amino acids in length.

6. An immunogenic composition as claimed in claim 4, wherein the synthetic peptide is about 10 to 100.

7. An immunogenic composition as claimed in claim 4, wherein the synthetic peptide is about 20 to 100 amino acids in length.

8. An immunogenic composition as claimed in claim 4, wherein the synthetic peptide is about 10 to 50 amino acids in length.

9. An immunogenic composition as claimed in claim 5, wherein the synthetic peptide is about 20 to 25 amino acids in length.

10. An immunogenic composition as claimed in claim 1 wherein the group A Streptococcus atmB putative lipoprotein comprises the amino acid sequence of SEQ ID NO. 1 or 2.

11. An immunogenic composition as claimed in claim 1, further comprising one or more other regions of group A Streptococcus antigens selected from the group consisting of regions of penicillin-binding proteins (PBPs), TdcF, CoaA, Spy1674, Spy1784 and Spy1733.

12. A vaccine comprising the composition as claimed in claim 1 and a pharmaceutically acceptable carrier, excipient, or diluent.

13. A vaccine comprising the composition as claimed in claim 11 and a pharmaceutically acceptable carrier, excipient, or diluent.

14. A method of inhibiting or reducing the growth of group A Streptococcus in blood, reducing phagocytic resistance, or a combination thereof in a subject, the method comprising administering an effective amount of the immunogenic composition as claimed in claim 1.

15. A method of immunizing a human against infection by group A Streptococcus by administering to the human an effective amount of the vaccine as claimed in claim 12.

16. A method for treating or preventing a GAS disease in a subject comprising administering to a subject in need thereof the composition as claimed in claim 1.

17. A method for stimulating or enhancing in a subject production of antibodies directed against a region of a group A Streptococcus atmB putative lipoprotein, the method comprising administering to the subject a vaccine as claimed in claim 12 in a dose effective for stimulating or enhancing production of the antibodies.

18. A method for diagnosing a GAS disease in a subject comprising:
(a) detecting in a sample from the subject one or more GAS markers wherein the GAS markers comprise group A Streptococcus atmB putative lipoprotein and polynucleotides encoding a group A Streptococcus atmB putative lipoprotein, and optionally one or more of penicillin-binding proteins (PBPs), polynucleotides encoding PBPs, TdcF, polynucleotides encoding TdcF, CoaA, polynucleotides encoding CoaA, Spy1674, polynucleotides encoding Spy1674, Spy1784, polynucleotides encoding Spy1784, Spy1733, and polynucleotides encoding Spy1733 and
(b) comparing the detected amounts of GAS markers with amounts detected for a standard.

19. A method as claimed in claim 18, the method comprising:
(a) contacting the sample with one or more antibody that specifically binds to a region of a group A Streptococcus atmB putative lipoprotein or parts thereof; and
(b) detecting in the sample amounts of group A Streptococcus atmB putative lipoprotein that bind to the antibody relative to a predetermined standard or cut-off value, and therefrom determining the presence or absence of the GAS disease in the subject.

20. A method as claimed in claim 19, the method comprising detecting penicillin-binding proteins (PBPs), TdcF, CoaA, Spy1674, Spy1784 and/or Spy1733.

21. A method as claimed in claim 18, the method comprising detecting one or more polynucleotides encoding a group A Streptococcus atmB putative lipoprotein in the sample from the subject and relating the detected amount to the presence of a GAS disease.

22. A method as claimed in claim 21, further comprising detecting polynucleotides encoding penicillin-binding proteins (PBPs), TdcF, CoaA, Spy1674, Spy1784 and/or Spy1733.

23. A method for diagnosing a GAS disease in a patient, the method comprising:
(a) detecting in a sample from the patient antibodies against group A Streptococcus atmB putative lipoprotein or parts thereof; and
(c) comparing the detected amount with an amount detected for a standard.

24. A method as claimed in claim 23, further comprising detecting antibodies to one or more of penicillin-binding proteins (PBPs), TdcF, CoaA, Spy1674, Spy1784 and Spy1733.

25. A kit for carrying out a method as claimed in claim 22.
